(19) United States
(12) Patent Application Publication
(10) Pub. No.: US 2026/0110021 A1
(43) Pub. Date: Apr. 23, 2026

(54) NUCLEIC ACID ANALYSIS

(71) Applicant: BIOFIDELITY LTD, Cambridge (GB)

(72) Inventors: Robert OSBORNE, Cambridge (GB); Magdalena STOLAREK-JANUSZKIEWICZ, Cambridge (GB); Barnaby William BALMFORTH, Cambridge (GB)

(21) Appl. No.: 19/118,748

(22) PCT Filed: Oct. 19, 2023
(86) PCT No.: PCT/IB2023/060587

§ 371 Date: Apr. 4, 2025

Related U.S. Application Data
(60) Provisional application No. 63/380,107, filed on Oct. 19, 2022.

(51) Int. Cl. C12Q 1/6855

(52) U.S. Cl. CPC C12Q 1/6855

(57) ABSTRACT

Provided herein are compositions and methods for the analysis of nucleic acids. In particular, provided herein are compositions and methods employing differential complementarity of probe nucleic acid molecules to target nucleic acids molecules to selectively isolate and detect targets or probes as an indication of the presence of and/or amount of a particular target nucleic acid molecule in a sample.

### CROSS-REFERENCE TO RELATED APPLICATIONS

The present application claims priority to United States Provisional Patent Application Ser. No. 63/380,107, filed Oct. 19, 2022, the disclosure of which is herein incorporated by reference in its entirety.

### SEQUENCE LISTING

The text of the computer readable sequence listing filed herewith, titled “41379-601_SEQUENCE_LISTING”, created Oct. 18, 2023, having a file size of 24,024 bytes, is hereby incorporated by reference in its entirety.

### FIELD OF THE INVENTION

Provided herein are compositions and methods for the analysis of nucleic acids. In particular, provided herein are compositions and methods employing differential complementarity of probe nucleic acid molecules to target nucleic acids molecules to selectively isolate and detect targets or probes as an indication of the presence of and/or amount of a particular target nucleic acid molecule in a sample.

### BACKGROUND

Targeted detection of low frequency variants in a pool of wild-type molecules is clinically important for early detection of cancer, monitoring of cancer progression, targeting of cancer therapies, non-invasive prenatal testing, monitoring of T-cell populations that target particular (neo-)antigens, and for early warning of organ transplant rejection. A combination of hybridisation-capture and next-generation sequencing (NGS) is the most commonly applied method for targeted and multiplex detection of low frequency variants but has several suboptimal characteristics. In general, hybridisation capture enriches for target regions of interest but not for variant molecules. This results in the vast majority of sequencing reads deriving from wild-type rather than variant molecules. This is wasteful, adds cost, and makes it difficult to detect rare variants against the background of errors from storage, library preparation and sequencing. This results in NGS having insufficient specificity for routine detection of variants below ˜0.1%. Modified library preparation methods (such as duplex sequencing) can increase specificity, yielding accurate sequencing data from single molecules. Unfortunately, methods like duplex sequencing also reduce sensitivity (for example, by modifying library preparation methods to avoid end-repair and by deliberately imposing molecular bottlenecks). The method described here allows preferential selection of probe oligonucleotides that differentiate a desired target nucleic acid from a non-desired target nucleic acid. Such probes are analysed, for example by sequencing, to indicate the presence of and/or amount of a corresponding target nucleic acid in a sample. This both reduces the number of sequencing reads that are required and allows multiplexed detection of low frequency variant molecules below the current limit of detection of NGS. In addition, a single base change is ‘converted’ into a ‘tag’ (consisting of multiple bases) which allows for error-detection and error-correction.

### SUMMARY

Provided herein are compositions and methods for the analysis of nucleic acids. In particular, provided herein are compositions and methods employing differential complementarity of probe nucleic acid molecules to target nucleic acids molecules to selectively isolate and detect targets or probes as an indication of the presence of and/or amount of a particular target nucleic acid molecule in a sample.

In some embodiments, nucleic acid from a sample is processed to generate a library. If the target is RNA, it may be converted to DNA prior to preparation of the library. Nucleic acid from the sample is fragmented and subjected to library preparation. Any of a wide variety of library preparation techniques may be used (e.g., generated by transposases). In some embodiments, one or more adapters and/or unique molecular identifiers (UMIs) is added to one or both ends of fragmented DNA. In some embodiments, fragmented DNA is end-repaired, A-tailed, and ligated to an adaptor. In some embodiments, the adapters employed differ from any sequencing adapters that are employed in later detection steps (e.g., if P5 and P7 Illumina sequencing adapters are used, they are not employed in the library preparation). In some embodiments, the library is amplified using primers comprising a capture moiety (e.g., biotin). In some embodiments, the capture moiety is at the 5′ end of the primer. In some embodiments, the capture moiety is positioned internally in the primer or near or at the 3′ end. The primers hybridize to the adapter sequence and generate amplicons having a capture moiety. In some embodiments, the library is denatured and captured on a solid surface (e.g., beads) that interacts with the capture moiety (e.g., streptavidin beads).

In some embodiments, probe oligonucleotides are provided that are differentially complementary to desired target nucleic acids relative to non-desired nucleic acids. For example, in some embodiments, the probes have greater complementarity to desired nucleic acids relative to non-desired nucleic acids. In some embodiments, the probes have less complementary to desired nucleic acids relative to the non-desired nucleic acids. For example, where a target nucleic acid comprises a specific polymorphism, the probe may be perfectly complementary to the target nucleic acid at the position of the polymorphism and mismatched to a corresponding nucleic acid (e.g., wild-type nucleic acid) not having the polymorphism; or the probe may be perfectly complementary to the wild-type nucleic acid and mismatched to a nucleic acid having the polymorphism.

In some embodiments, one or more probes designed to analyse one or more target nucleic acids are exposed to the surface-captured library under conditions that support hybridization between probes and their respective complementary pairs. Non-hybridized probes are washed away under appropriate conditions (e.g., temperature, pH, buffer composition) to remove non-specific binding probes. Probes that are hybridized to the surface-bound library fragments are exposed to selective cleavage conditions that cause probes bound to desired sequences to be differentially modified compared to probes bound to non-desired sequences. In some embodiments, the probes hybridized to desired target nucleic acid sequences are cleaved or partially digested such that they are separated or separable (e.g., based on changes in temperature, pH, etc.) from the surface-bound fragment, whereby probes hybridized to non-desired sequences are not modified and remain hybridized to surface-bound fragment under the reaction conditions employed to separate the probes that were hybridized to the desired sequences. Such probes are isolated and analysed (e.g., sequenced). In some embodiments, the probes hybridized to the undesired sequences are modified such that they are separated or separatable and the probes hybridized to the desired sequences remain associated with the surface-bound fragments. Such probes can be subsequently released from hybridization, isolated, and analysed (e.g., sequenced).

In some embodiments, probes are made resistant to digestion (e.g., through mismatches, backbone or base modifications, etc.) and are selectively ‘activated’ based on their differential hybridization to different fragments (e.g., alleles), allowing the probes to be subsequently digested. In some embodiments, the activation step provides a nick or gap in the probe.

In some embodiments, activation occurs using one or more restriction endonucleases. In some embodiments, the restriction endonuclease is a methylation/modification-specific endonuclease. In some embodiments, activation occurs using a Flap endonuclease. In some embodiments, the activation occurs using a mismatch repair enzyme or mismatch-specific endonuclease (e.g., CelI, S1, T7E1, SURVEYOR nuclease). In some embodiments, activation occurs using an RNase enzyme (e.g., RNaseH2). In some embodiments, activation occurs using a CRISPR/Cas systems (e.g., including use of Cas enzymes or mutants that cleave only one strand). In some embodiments, activation occurs using an argonaute family enzyme (e.g., Ago1, Ago2, Ago3, Ago4, Hili, Hiwi, Hiwi2, Hiwi3, aubergine, PIWI, Ago5, Ago6, Ago7, Ago8, Ago9, Ago10, Alg-1, Alg-2, etc.). In some embodiments, activation occurs using chemical cleavage of mismatch (CCM) (e.g., hydroxylamine+potassium permanganate followed by piperidine).

In some embodiments, digestion of the probe is selective based on differential hybridization to the fragment. In some embodiments, digestion of the probe employs a general digestion method that is selective for activated probes. In some embodiments, digestion is 3′-5′ in direction. In some embodiments, digestion is 5′-3′ in direction. In some embodiments, digestion is via internal cleavage of the probe. In some embodiments, digestion comprises pyrophosphorolysis (cleavage in which inorganic phosphate is the attacking group). In some embodiments, a diphosphohydrolase enzyme (e.g., Apyrase) or phosphatase (e.g., Antarctic phosphatase, shrimp alkaline phosphatase) is provided in the methods or compositions of the invention. The diphosphohydrolase enzyme hydrolyses released nucleotides from the pyrophosphorolysis reaction, maintaining optimal pyrophosphorolysis reaction conditions. In some embodiments, inorganic pyrophosphatase enzyme is provided in the methods or compositions of the inventions. Inorganic pyrophosphatase removes pyrophosphate ions after pyrophosphorolysis and before subsequent steps where the presence of pyrophosphate ions may be undesired or suboptimal.

In embodiments where 3′-5′ digestion is desired, a 3′-5′ exonuclease is employed. Such exonucleases include, but are not limited to, Exonuclease I (ExoI) (e.g., E. coli ExoI), Exonuclease T (ExoT), Exonuclease VII (ExoVII), Exonuclease III (ExoIII), and DNS.

In some embodiments where 3′-5′ digestion is desired, a polymerase having 3′-5′ exonuclease activity is employed (e.g., proofreading polymerases, e.g., PHUSION (Thermo Fisher), Q5 (New England Biolabs), KAPA HIFI (Roche), KOD DNA polymerase).

In some embodiments where 5′-3′ digestion is desired, a 5′-3′ exonuclease is employed (e.g., Lambda Exonuclease, RecJf, T7 exonuclease, Exonuclease V (ExoV), Exonuclease VIII (ExoVIII), T5 exonuclease). Alternatively, a DNA polymerase with 5′-3′ exonuclease may be employed.

For internal cleavage, any of the “activation” methods described above can be used as a standalone digestion method. In some embodiments, the resulting fragments from the cleaved probe have a lower melting temperature (Tm) than the intact probe, allowing selective denaturation from the fragment by an increase in temperature, pH etc. In some embodiments, the activation step itself is sufficient to permit a probe to melt from the fragment without requiring reaction condition changes.

In some embodiments, where pyrophosphorolysis (PPL) is desired, a polymerase exhibiting pyrophosphorolysis activity is employed. In some embodiments, probes that were hybridized to a mutant sequence (probes that are complementary to the mutant and mismatched verse the corresponding wild-type sequence) are released into the supernatant, while probes that were hybridized to a wildtype sequence remain hybridized to an immobilized library molecule. In some embodiments probes that were hybridized to a wildtype sequence are released into the supernatant while probes that were hybridized to a mutant sequence remain hybridized to an immobilized library molecule. In some embodiments, the supernatant is collected, which includes an enriched fraction of PPLed probes, while in some embodiments, the supernatant is removed and any probes retained on an immobilized library molecule are subsequently released and analyzed. These probes are then processed for sequencing via 3′ adaptor ligation. In some embodiments, the method for ligating 3′ adaptors uses a 5′ preadenylated adaptor sequence, which is then ligated by APP or T4 DNA ligases. Alternatively, commercial methods/kits are available (e.g., use of TdT or kits from Swift Biosciences or SMART-Seq (Takara); adenylation kit from New England Biolabs). The ligated molecules can be amplified (e.g., by PCR) prior to sequencing. In some embodiments, the adaptor has a phosphate or is 5′ phosphorylated (e.g., using T4 Polynucleotide kinase). In some embodiments, the adaptor is protected on its 3′ end from self-ligation (for example using ddC).

In some embodiments, the library fragments are hybridized to probes prior to their capture on the solid surface. The hybridization complexes are subsequently captured onto a solid support.

In some embodiments, probe oligonucleotides comprise a sequencing adapter (e.g., P5 adapter) at their 5′ ends and the 3′ ligation adds a second sequencing adapter (e.g., P7′ adapter). In some embodiments, the reverse polarity is used (probe initially provided with a P7 adapter and a P5′ adapter is added by 3′ ligation). Ideally, this would be configured so that low complexity sequences are less likely during the first few bases of sequencing read 1 (this is when the Illumina crosstalk calibration takes place). In some embodiments, the probes do not initially include any sequencing adapter sequences. Sequencing adapters can be added via ligation and/or amplification (e.g., using a primer having a 5′ adapter sequence and a 3′ sequences complementary to a known sequence on the probe. An example of this approach is illustrated in FIG. 1.

In some embodiments, the adaptor sequence is further modified. For example, the adapter sequence may further comprise a UMI and/or an MID. In some embodiments, this is followed by a clean-up step before amplification.

In some embodiments, the probe, following modification (e.g., cleavage and/or digestion) is enriched either before, or after, amplification e.g., through gel purification, Solid-phase reversible immobilization (SPRI) bead clean up, or HPLC.

Inclusion of a UMI in the probe sequence finds use to identify reads that belong to the same digested probe. This information may be used to derive a consensus sequence of the digested probes, thereby removing errors that occur during PCR. In addition, this information may be used to count probe molecules, thereby providing information on the copy number of variant molecules.

Depending on the reaction schema, if the original library is PCR amplified, it may not be possible to determine whether a probe has been digested against an original template molecule or a copy of an original template molecule. If the original library is not PCR amplified, or linearly amplified, it is possible to identify probes that have been digested against one, or other, of the strands. This information finds use for error-correction/detection during data analysis. In some embodiments, the adapters added to the library molecules comprise a UMI and the probe is extended along the library molecule prior to ligation of an adapter, allowing determination of the original template molecule from which the library molecule to which the probe hybridized was derived.

Depending on the nature of the cleavage/digestion mechanism employed, one may obtain a distribution of different stop points from the digested probes. It is possible to compare the distribution of stop points for first sample (e.g., including a mutant sequence) versus second sample (e.g., fully wildtype). The presence of the mutation can then be inferred by the distribution of stop points. It is contemplated that different mutations have different distributions of stop points (i.e., a stop point “fingerprint”). For example, one can identify a C>T mutation from a C>A mutation at the genomic position using the stop point information.

In some embodiments, probes are hybridized to targets (or amplicon copies thereof) and digested prior to capture. For example, in some embodiments, nucleic acid from a sample is fragmented, end-repaired, A-tailed, and adaptor ligated. The library is amplified, for example, via PCR. The library is denatured and hybridized to probes. Probes are optionally activated and are digested (e.g., pyrophosphorolysed). In some embodiments, probes that are hybridized to a mutant sequence are released into the supernatant. In contrast probes that are hybridized to a wildtype sequence remain hybridized to a library molecule. There will typically be a large fraction of unhybridized ‘background’ probes. The reaction mixture is depleted of full-length (or close to full-length), non-digested probes. This can be accomplished, for example, by addition of a second biotinylated probe that is complementary to the digested end of probes. Undigested probes will bind to the biotinylated probe while digested probes will not. The complexes are captured on streptavidin beads. The supernatant is collected, which includes an enriched fraction of the digested probes. Alternatively, digested probes can be captured on a solid support. These digested probes are then processed for sequencing (e.g., via 3′ adaptor ligation). The ligated molecules can optionally be amplified. And example of this approach is illustrated in FIG. 2.

In some embodiments, a mutation agnostic methodology is employed. Fragmented nucleic acid is subject to end-repair, A-tailing, and adaptor ligation. The adaptors include a double-stranded stem and a 5′ sequence adapter (e.g., P7). The 3′ of the stem includes a biotin moiety. The library is denatured and immobilized onto streptavidin beads. Probes are hybridized to the immobilized library. In some embodiments, probes match wild-type sequence. The library is optionally washed at this point to remove non-hybridized probe sequences. The library undergoes optional activation and digestion, followed by washing. Probes hybridized to wildtype molecules are digested and released into the supernatant. Probes hybridized to mutant molecules are either not digested or are less digested, remaining hybridized to their target. Where PPL is employed as the digestion mechanism, this typically results in a 3′ mismatch between the digested probe and the variant sequence. The 3′ mismatch is removed using a single-stranded DNA exonuclease (e.g., S1 nuclease or exonuclease I), a mismatch repair endonuclease (e.g., CelI), or a 3′ flap endonuclease. If S1 is used it may be beneficial to protect 5′ single-stranded regions of both the probe (5′ flap) and the target. Alternatively, one can use a DNA polymerase with 3′ to 5′ exonuclease activity. The 3′ end of the probe is then extended. Note that the use of a DNA polymerase with both 5′ to 3′ polymerase and 3′-5′ exonuclease activities can perform both the 3′ mismatch removal and 5′-3′ extension reaction in a single step. The probe is then amplified using sequencing primers (e.g., P5 and P7) in an amplification reaction before sequencing.

The orientation of sequencing adapters can be reversed. The stem′ region could include a 3′ block or a mismatch to one of the sequence adapters (e.g., the P7 sequence). This reduces P7-P7′ amplicons.

This approach does not require knowledge of the mutation site and nucleotide change. Individual samples are readily identifiable through their MIDs. One fragmentation breakpoint is available to help identify individual input molecules. An example of this methodology is illustrated in FIG. 3.

In an alternative mutation agnostic method, fragmented nucleic acid is subject to end-repair, A-tailing, and adaptor ligation. The adaptors include a double-stranded stem and a 3′ reverse complement sequencing adapter sequence (e.g., 3′ P7′) including a biotin moiety. The library is denatured and immobilized onto streptavidin beads. Probes are hybridized to the immobilized library. In some embodiments, probes match wild-type sequence. The library is optionally washed at this point to remove non-hybridized probe sequences. The library undergoes optional activation and digestion, followed by washing. Probes hybridized to wildtype molecules are digested and released into the supernatant. Probes hybridized to mutant molecules are either not digested or partially digested, but remain hybridized to their target. Where PPL is employed as the digestion mechanism, this typically results in a 3′ mismatch between the PPLed probe and the variant sequence. The 3′ mismatch is removed using a single-stranded DNA exonuclease (e.g., S1 nuclease or exonuclease I), a mismatch repair endonuclease (e.g. CelI), or a 3′ flap endonuclease. The 3′ end of the probe is then extended. Note that the use of a DNA polymerase with both 5′ to 3′ polymerase and 3′-5′ exonuclease activities can perform both the 3′ mismatch removal and 5′-3′ extension reaction in a single step. A second adaptor is then ligated to the resulting extension product. In some embodiments, this includes a 5′ P5 sequence. Once ligated a molecule is generated having the structure: 5′-P7-insert-P5′-3′ (or if different adaptors are used, 5′-P5-insert-P7′-3′). Excess adaptor can then be removed by washing. The enriched library is amplified by PCR. An example of this approach is shown in FIG. 4A.

In some embodiments, a hairpin linker is used to connect the probe and the fragment of interest. In this case the P5 sequence is added via the probe rather than during the second ligation step (see FIG. 4B).

In these embodiments it is possible to deploy a full range of molecular counting techniques, including use of fragmentation breakpoints, UMIs and orientation of reads relative to adaptors (to identify individual input strands). The method is also amenable to duplex sequencing.

If there is a desire to amplify the initial library, in some embodiments, the library could be PCR amplified with a Y-shaped adaptor tagged library followed by selecting one of the strands based on the orientation of the adaptor sequences. In some embodiments, these are 3′ biotinylated via non-templated DNA polymerase extension or terminal transferase. Alternatively, molecules can be hybridised to streptavidin beads preloaded with appropriate capture sequences. In this case, a probe would only pull out one strand of an original template molecule. Where there is a desire to sequence both strands of the same molecule, two probes may be employed, each targeting a different strand.

In another alternative mutation agnostic technique, library construction is similar to the mutation agnostic described immediately above. Probes are hybridized to the library. Probes have a sequencing adapter tail (e.g., P5 tail). The library undergoes optional activation and digestion (e.g., PPL). Probes hybridized to wildtype molecules are digested and released into the supernatant. Probes hybridized to mutant molecules are not digested or digested less and remain hybridized to their target. Where PPL is employed as the digestion mechanism, this typically results in a 3′ mismatch between the PPLed probe and the variant sequence. The 3′ mismatch is removed using a single-stranded DNA exonuclease (e.g., S1 nuclease or exonuclease I), a mismatch repair endonuclease (e.g., CelI), or a 3′ flap endonuclease. Alternatively, a DNA polymerase with 3′ to 5′ exonuclease activity is used.

The 3′ end of the probe is then extended. The use of a DNA polymerase with both 5′ to 3′ polymerase and 3′-5′ exonuclease activities can perform both the 3′ mismatch removal and 5′-3′ extension reaction in a single reaction. In some embodiments, the extension is performed in the presence of biotin labelled dNTPs. The resulting biotinylated extension product has a 5′-P5-insert-P7′-3′ sequence and is captured onto a solid support. Any uncaptured molecules are then removed and the biotinylated extension product is PCR amplified using P5 and P7 primers and sequenced. The original orientation of the molecule can be ascertained by the orientation of adaptor sequences relative to the insert. Two probes are used to recover both original template strands, allowing duplex sequencing. The same would be true if the library is PCR amplified. An example of this approach is shown in FIG. 5.

In some embodiments, the methods are used for detection of gene fusions. Gene fusions are an increasingly important class of biomarker and are hard to detect efficiently via DNA sequencing. DNA-based NGS assays miss large numbers of these variants due to the enormous number of potential DNA changes that can lead to fusions at the RNA level. A further challenge in detecting gene fusions is that there may be many partner genes/exons to which the gene in question is fused. In an ideal world, fusions would therefore be detected from RNA in a manner that is independent of the partner gene, is performed simultaneously with DNA-based somatic variant analysis, and is enriched for variant sequences to maximise sequencing efficiency.

In some embodiments the RNA in the sample is first transcribed to DNA prior to application of the methods described herein. In some embodiments the methods described herein are applied directly to RNA molecules in the sample without transcription.

Using the ‘mutation agnostic’ methodologies described above, gene fusions can be detected. Probes perfectly matching the wild-type allele and crossing the exon/exon boundary would have 3′ mismatch to any fusion variants, independent of the partner, enabling enrichment.

Mutation specific methodologies may also be employed to detect gene fusions. Such methods are more complicated because the partner gene, and so the mutant sequence, is not known. This challenge is overcome by using multiple different probes, covering every combination of bases at the 3′ end of or in the terminal few bases at the 3′ end, except for the combination giving perfect complementarity to the WT sequence. An example of this approach is illustrated in FIG. 6.

An alternative embodiment employs a probe that perfectly matches the conserved region of the target sequence (i.e., it does not cross the exon/exon boundary) combined with a ‘blocking’ oligonucleotide which spans the exon/exon boundary and overlaps the hybridization region of the 3′ end of the probe. This blocking oligonucleotide may be designed such that it will stably hybridize only to wildtype molecules in the conditions employed, creating a mismatched flap at the 3′ end of probe hybridised to wildtype molecules. No such flap is formed with non-wild-type fusion partners. Probe molecules hybridized to wildtype and variant molecules can then be differentially activated and/or digested. An example of this approach is illustrated in FIG. 7. In some embodiments, the blocking oligonucleotide is protected from digestion (e.g., PPL; e.g. via mismatches, modifications, RNA bases etc.). A further, non-PPL-based approach uses short hybridisation regions in the probes, followed by extension of the probes using a polymerase with no strand displacement or 5′-3′ exonuclease activity. Probes hybridised to WT molecules are blocked from extension by the blocking oligonucleotide and melt during subsequent steps. In this embodiment the hybridization region of the blocking oligonucleotides in the target sequence need not overlap the hybridization region of the probes.

The above approaches also find use for detection of indels from DNA.

With any of the fusion or indel methodologies, detection may occur via a route that employs activation and/or digestion with techniques that employ probe sequencing (e.g., those approaches described elsewhere herein) or via techniques that do not employ probe sequencing (e.g., those approaches described in PCT/IB2022/000217 or U.S. Prov. Appln. Ser. No. 63/380,105, each of which is herein incorporated by reference in its entirety). For example, in the latter instance the probe molecules may be captured onto a solid support, and variant molecules enriched for subsequent analysis through differential digestion of probes hybridized to variant versus wildtype molecules.

In some embodiments, the present systems and methods use selective digestion as a method of enriching for or depleting nucleic acid sequences. In some embodiments, the digestion reaction relies on complementarity between hybridised strands, and only digests strands having a particular characteristic (e.g., a specific sequence or structure). The reaction selectively shortens or digests certain sequences having the particular characteristic, while leaving sequences not having the characteristic undigested or less digested. The reaction can be performed such that molecules hybridized to the shortened or more digested sequences are recovered and analysed. Alternatively, the reaction can be performed such that less shortened or more digested sequences are analysed. Alternatively, the reaction can be performed so that the sequences which have not undergone any digestion are analysed.

In some embodiments, provided herein is a hybridisation/pyrophosphorolysis method. These methods harness the double-strand specificity of pyrophosphorolysis; a reaction which will not proceed efficiently with single-stranded oligonucleotide substrates or double-stranded substrates which include mismatches.

For example, in some embodiments, provided herein are methods that comprise contacting a sample (e.g., containing two or more different nucleic acid molecules) with a probe and pyrophosphorolysis reaction reagents and enriching for or depleting a first nucleic acid molecule relative to a second nucleic acid molecule based on different complementarity of the probe to the first and second nucleic acid molecules, resulting in different levels of pyrophosphorolysis of the probe when hybridized to the first and second nucleic acid molecules.

In some embodiments, the digestion continues until probe lacks sufficient complementarity with the sequence for the pyrophosphorolysing enzyme to bind or for the pyrophosphorolysing reaction to continue. This typically occurs when there are between 6 and 20 complementary nucleotides remaining between the sequence and probe. In some embodiments, this occurs when there are between 6 and 40 complementary nucleotides remaining. In some embodiments, this occurs when there are between 4 and 100 or more complementary nucleotides remaining (depending on buffer conditions and the PPL enzyme used)

Without being constrained by theory, there are a number of different ways in which the pyrophosphorolysis reaction may be stopped. If the pyrophosphorolysis enzyme has the ability to ‘read ahead’, the digestion may stop 3′ of a mismatch or base modification. Such activity has been observed in archaeal DNA polymerases. In some embodiments, the pyrophosphorolysis reaction may stop due to the presence of a modification in the backbone of the probe. This modification may be a modified base. The base may be resistant to pyrophosphorolysis. This modification may be a chemical backbone modification. In some embodiments, the pyrophosphorolysis reaction may stop due to the presence of mismatch in the probe. The location of this mismatch may be purposefully designed such that digestion halts at this defined point. In some embodiments, the temperature of the reaction mixture may be increased to heat-inactivate the pyrophosphorolysis enzyme. In some embodiments, the temperature is increased to cause the probe-target duplex to melt apart. In some embodiments, any reagent that could cause the inactivation of the pyrophosphorolysis enzyme may be added to the reaction mixture. In some embodiments, the pH concentration may be modified to inactivate pyrophosphorolysis enzyme. In some embodiments, the salt concentration may be modified to inactivate pyrophosphorolysis enzyme. In some embodiments, the detergent concentration may be modified to inactivate pyrophosphorolysis enzyme. In some embodiments, the ion concentration may be modified to inactivate pyrophosphorolysis enzyme. The person skilled in the art will appreciate that there are numerous further ways in which an enzyme catalysed reaction may be brought to a halt and the above disclosure is not intended to limit the scope of the invention (e.g., use of TIPP).

Suitably, pyrophosphorolysis is carried out in the reaction medium at a temperature in the range 20 to 90° C. in the presence of at least a polymerase exhibiting pyrophosphorolysis activity and a source of pyrophosphate ion. Further information about the pyrophosphorolysis reaction as applied to the digestion of polynucleotides can be found for example in J. Biol. Chem. 244 (1969) pp. 3019-3028, herein incorporated by reference in its entirety.

In some embodiments, the pyrophosphorolysis step is driven by the presence of a source of excess polypyrophosphate, suitable sources including those compounds containing 3 or more phosphorous atoms.

In some embodiments, the pyrophosphorolysis step is driven by the presence of a source of excess modified pyrophosphate. Suitable modified pyrophosphates include those with other atoms or groups substituted in place of the bridging oxygen, or pyrophosphate (or polypyrophosphate) with substitutions or modifying groups on the other oxygens. The person skilled in the art will understand that there are many such examples of modified pyrophosphate which would be suitable for use in the current invention, a non-limiting selection of which are:

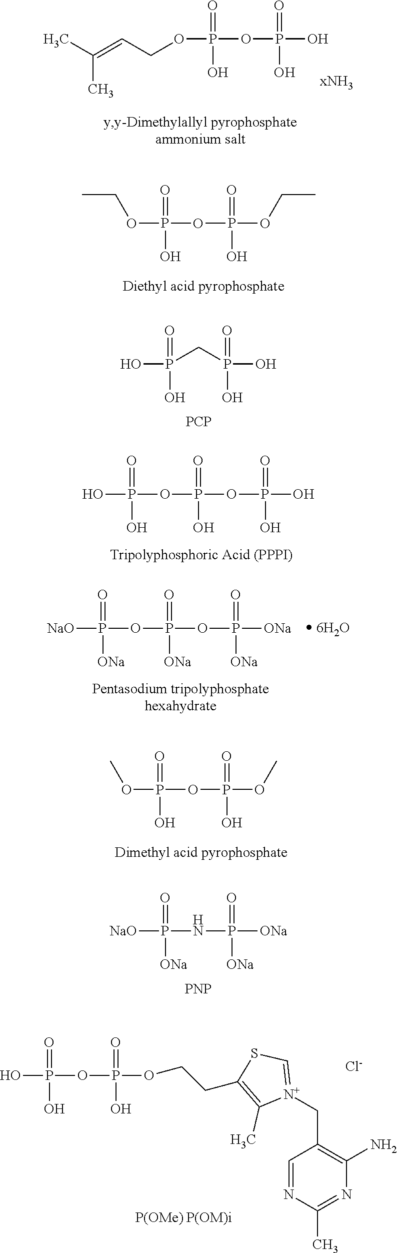

In one preferred embodiment, the source of pyrophosphate ion is PNP, PCP or Tripolyphosphoric Acid (PPPi).

Further, but not limiting, examples of sources of pyrophosphate ion for use in the pyrophosphorolysis step may be found in WO2014/165210 and WO00/49180, herein incorporated by reference in their entireties.

In some embodiments, the source of excess modified pyrophosphate can be represented as Y—H wherein Y corresponds to the general formula (X—O)₂P(═B)—(Z—P(═B)(O—X))ₙ— wherein n is an integer from 1 to 4; each Z— is selected independently from —O—, —NH— or —CH₂—; each B is independently either O or S; the X groups are independently selected from —H, —Na, —K, alkyl, alkenyl, or a heterocyclic group with the proviso that when both Z and B correspond to —O— and when n is 1 at least one X group is not H.

In some embodiments, Y corresponds to the general formula (X—O)₂P(═B)—(Z—P(═B)(O—X))ₙ— wherein n is 1, 2, 3 or 4. In another embodiment, the Y group corresponds to the general formula (X—O)₂P(═O)—Z—P(═O)(O—H)— wherein one of the X groups is —H. In yet another preferred embodiment, Y corresponds to the general formula (X—O)₂P(═O)—Z—P(═O)(O—X)— wherein at least one of the X groups is selected from methyl, ethyl, allyl or dimethylallyl.

In an alternative embodiment, Y corresponds to either of the general formulae (H—O)₂P(═O)—Z—P(═O)(O—H)— wherein Z is either —NH— or —CH₂— or (X—O)₂P(═O)—Z—P(═O)(O—X)— wherein the X groups are all either —Na or —K and Z is either —NH— or —CH₂—.

In another embodiment, Y corresponds to the general formula (H—O)₂P(═B)—O—P(═B)(O—H)— wherein each B group is independently either O or S, with at least one being S.

Specific examples of preferred embodiments of Y include those of the formula (X1-O)(HO)P(═O)—Z—P(═O)(O—X2) wherein Z is O, NH or CH₂ and (a) X1 is γ,γ-dimethylallyl, and X2 is —H; or (b) X1 and X2 are both methyl; or (c) X1 and X2 are both ethyl; or (d) X1 is methyl and X2 is ethyl or vice versa.

In some embodiments, the probe has greater complementary to the target region of the first nucleic acid molecule than it does to a corresponding target region of a second nucleic acid in the sample. In some embodiments, the probe has lesser complementary to the target region of the first nucleic molecule than it does to a corresponding target region of a second nucleic acid in the sample. In some embodiments, the first nucleic acid and the second nucleic acid differ by a sequence variation (e.g., a point mutation, a deletion, an insertion, multinucleotide change, fusion, etc.). In some embodiments, the probe includes a sequence that is perfectly complementary to the target region of the sequence of greater complementarity and has one or more mismatches to a sequence variation found in the corresponding target region of the sequence of lesser complementarity. In some embodiments, the probe contains one or more mismatches to the target regions of both the first and second nucleic acid molecules, but contains more mismatches to the target region of the nucleic acid of lesser complementarity. In particular, the probe is designed such that the cleavage of the probe differs when the probe is hybridized to a first nucleic acid relative to a second nucleic acid, permitting selective enrichment, depletion, isolation, and/or analysis of the first nucleic acid relative to the second nucleic acid or of probes hybridized thereto.

Probes may be provided with one or more components that are removed prior to or during cleavage. For example, probes may be provided with a non-complementary flap or other blocking group at their 3′ end or 5′ end that prevents digestion or polymerase reaction until the blocking group is removed. The blocking group may be removed by any suitable mechanism (e.g., enzymatic cleavage, chemical reaction, temperature shift, etc.). Any suitable blocking group may be employed, including, but not limited to, include of phosphorothioate bonds, use of modified bases (e.g., 2′-O-Methyl, 2′ Fluoro, etc.), inverted or dideoxy nucleotides, phosphorylation, inclusion of spacers, and the like. The sequence of the probe that provides the differential cleavage products, when hybridized to distinct nucleic acid molecules, may be positioned at any suitable location in the initial probe. For example, a mismatch sequence may be positioned at the 3′ terminal base of the 3′ end of the probe. A mismatch sequence may be positioned internally in the probe at the 3′ end. A mismatch sequence may be positioned centrally in the probe. A mismatch sequence may be positioned within the 5′ half of the probe or at the 5′ end of the probe (e.g., the 5′ terminal base).

The 5′ or 3′ end of the probe may comprise a region (e.g., a 5′ or 3′ tail) that acts as an identifier (e.g., a sample identifier). Such a sequence finds use, for example, to selectively pull down captured molecules from a specific sample or specific region from a mixed sample. Such identifiers may find particular use in multiplex reactions where multiple different targets are undergoing reactions in the same sample or same reaction vessel.

In some embodiments, methods employ a combination of probes in the same sample preparation, some of which are designed to enrich for or deplete sequences containing variants (e.g., as described above) and some of which are designed to simply capture regions of interest (e.g., using any known hybridization/capture technology or approach). For example, in some embodiments, such combination methods find use where MSI or copy number variants are being analysed in the same sequencing run as somatic variants: the somatic variants are enriched while the genes/regions, used for MSI/CNV analysis, are captured via standard methods. One potential issue is that the standard hybridication/capture probes might be undesirably modified by enzymes and/or reagents used in the enrichment/depletion methodology. To prevent this, in some embodiments, the standard hybridization/capture oligonucleotides are made resistant to the modification enzymes/reagents. For example, the standard hybridization/capture oligonucleotides may be modified by use of RNA rather than DNA, use of modified bases or backbone modifications, or by introducing an intentionally mismatched region in the probe. There are some scenarios in which it may be beneficial to perform the probe hybridisation of standard and enrichment/depletion probes simultaneously and then subsequently separate them. In some embodiments, this is performed through the use of different attachment chemistries on the different probe types, or through different 5′ or 3′ sequences on the probes which can be differentially captured onto solid supports via hybridisation to complementary ‘linker’ oligos which themselves include attachment moieties (or are pre-linked to solid supports).

The analytes/sequences to which the method of the invention can be applied are those nucleic acids, such as naturally-occurring or synthetic DNA or RNA molecules, which include the target polynucleotide sequence(s) being sought. In some embodiments, the analytes/sequences will typically be present in an aqueous solution containing it and other biological material and, in some embodiments, the analytes/sequences will be present along with other background nucleic acid molecules which are not of interest for the purposes of the test. In some embodiments, the analytes/sequences are present in low amounts relative to these other nucleic acid components. In some embodiments, for example where the analyte is derived from a biological specimen containing cellular material, prior to performing the enrichment or depletion methods described herein, some or all of these other nucleic acids and extraneous biological material are removed using sample-preparation techniques such as filtration, centrifuging, chromatography or electrophoresis. In some embodiments, DNA or RNA molecules are included in a mixture comprising a sequencing library. The sequencing library may be derived from and/or include either or both single-stranded or double-stranded molecules and may include DNA and/or RNA. Library sequences may include modifications, such as adapters, unique molecule indexes (UMIs), primer binding sequences, or the like and may be prepare by any suitable process (e.g., tagmentation).

The compositions and methods of the invention may be employed against any type of sample, including, but not limited to environmental (e.g., water, soil, air, etc.) samples and biological samples. Biological samples may be from any source including plants, animals, infectious disease agents, and the like. Suitably, in some embodiments, the analytes/sequences are derived from a biological sample taken from a mammalian subject (especially a human patient) such as blood, plasma, sputum, urine, skin, biopsy or surgical resection. In some embodiments, the biological sample are subjected to lysis in order that the analytes/sequences are released by disrupting any cells present. In other embodiments, the analytes/sequences may already be present in free form within the sample itself; for example, cell-free DNA circulating in blood or plasma. The compositions and methods of the invention find particular use with historically challenging sample types that may have low allele fractions of the analyte of interest. Such samples include blood, urine, cytosponge-collected samples (e.g., oesophageal samples), bronchoalveolar lavage (BAL) derived samples, pleural fluid, and cerebrospinal fluid (CSF).

In some embodiments, samples are pooled samples. Pooled samples involve mixing multiple samples togethers in a batch where the pooled collection is tested. This approach increases the number of individual samples that can be tested using a more limited amount of resources. Pooled samples of interest include, but are not limited to, donated blood samples, agricultural samples, food samples, sperm samples, and biological samples tested for the presence of infectious disease agents (e.g., SARS-CoV-2, HIV, HCV, etc.). In some embodiments, the pooled sample is an environmentally collected sample (e.g., wastewater sample) that has, by the nature of its generation, pooled samples from multiple different sources. While pooling of samples may reduce the allele fraction of variants as the samples dilute each other, it can provide a dramatic increase in efficiency of screening. Because the technology provided herein enables detection at very low allele fractions, it is particularly well suited for analysis of pooled samples. In some embodiments, a fraction of each initial sample is pooled without use of barcodes or other complex preparation steps and the pooled sample is tested. If a positive result is obtained, remaining fractions of the unpooled samples may be tested individually.

Also provided herein are compositions (e.g., reagents, kits, reactions mixture, instruments, software) that find use with the methods described herein. For example, in some embodiments, provided here are compositions comprising one or more reagents necessary, sufficient, or useful for conducting a method as described herein. For example, in some embodiments, compositions comprise: one or more probes that comprises a sequence that is differentially complementary to a known first sequence and a known second sequence (e.g., perfectly complementary to a known first sequence but imperfectly complementary to a known second sequence); one or more reagents that selectively modify probes hybridized to a desired nucleic acid relative to probes hybridized to a non-desired nucleic acid; and/or one or more agents that cleave or digest probes. In some embodiments, the compositions further comprise a target nucleic acid isolation component that segregates target nucleic acid molecules. In some embodiments, the compositions comprise one or more solid supports. In some embodiments, the compositions comprise one or more buffers. In some embodiments, the solid support is a bead (e.g., magnetic or paramagnetic bead). In some embodiments, the composition further comprises one or more epigenetic modification sensitive or dependent restriction enzymes. In some embodiments, the composition further comprises one or more restriction endonucleases. In some embodiments, the composition further comprises one or more transposomes. In some embodiments, the composition comprises a Cas protein (e.g., Cas9). In some embodiments, the composition further comprises one or more transposases. In some embodiments, the composition further comprises one or more ligases. In some embodiments, the composition further comprises one or more blocking oligonucleotides. In some embodiments, the composition further comprises reagents for conducting an amplification (e.g., PCR), sequencing (e.g., next generation sequencing), or detection reaction. In some embodiments, the composition further comprises one or more molecular beacon probes. In some embodiments, the one or more molecular beacon probes are fluorescently labelled. In some embodiments, the composition further comprises components for the transcription of RNA into cDNA.

In some embodiments, the composition is a reaction mixture comprising a reaction, at a particular time point, of any of the methods described herein. In some embodiments, the reaction mixture comprises probe/nucleic acid hybridization complexes of the methods described herein. In some embodiments, the reaction mixture comprises captured nucleic acid molecules of the method described herein. In some embodiments, the reaction mixture comprises regions comprising concentrations of a desired target nucleic or probe (e.g., digested probe) that are higher or lower than the concentration of the desired target nucleic that was present in a sample that underwent a digestion reaction. For example, in some embodiments, provided herein are reaction mixtures comprising: a sample; reagents for modifying a probe hybridized to a target nucleic acid; a first nucleic acid molecule from the sample hybridized to a probe having a sequence, wherein a discrimination region of the probe is complementary to the first nucleic acid molecule; and a second nucleic acid molecule from the sample hybridized a probe having said sequence, wherein the discrimination region of the probe is not perfectly complementary to the second nucleic acid molecule.

Also provided herein are uses of the compositions (e.g., uses of the kits, uses of the reaction mixtures, uses of the reagents, uses of the instruments, uses of the software). For example, provided herein are uses of the composition for enriching or depleting a target nucleic acid in a sample.

In some embodiments, provided herein are devices and instruments that find use in the methods described herein. In some embodiments, the devices and instrument find use to collect and distribute samples into reaction vessels. In some embodiments, the devices and instruments provide reaction chambers for conducting the methods. In some embodiments, the devices and instruments provide multiple zones or regions (e.g., wells, channels, etc) for housing a reaction and/or for isolating enriched desired target nucleic acids or depleting desired target nucleic acids. In some embodiments, the devices and instruments find use to amplify or sequence nucleic acid molecules. In some embodiments, the devices and instruments find use to detect nucleic acid molecules. In some embodiments, the devices and instruments find use to receive or transmit information from a user. For example, the devices and instrument may comprise a user interface to receive user instructions and a display to visually present results to a user.

In some embodiments, provided herein are computing devices. The computing devices find use to control instruments or devices to facilitate the methods described herein. In some embodiments, the computing devices collect, analyse, and report data. In some embodiments, the computing devices comprise one or more processors that run a computer program. In some embodiments, the computing devices comprise non-transitory computer readable media (e.g., software) comprising instructions that direct a processor to carry out one or more of the computing steps.

In some embodiments, the method comprises analysing molecules with specific fragmentation profiles. In some embodiments, the capture comprises capturing a nucleic acid fragment with a probe that has sequence identity to a fragmentation breakpoint and adaptor sequences. In some embodiments, the capture can include capture of molecules with specific 5′ and 3′ breakpoints. In some embodiments, capture can include sequential hybridisation and capture of each breakpoint. In other embodiments, capture can include hybridisation and capture using multiple probes to capture molecules with specific 5′ and 3′ fragmentation breakpoints. Fragmentation patterns contain information on nucleosomal organisation, chromatin structure, gene expression, and nuclease content of the tissue of origin, resulting in characteristic signatures in the form of fragment size, nucleotide motifs at the fragment ends, single-stranded jagged ends, and the genomic locations of the fragmentation endpoints. For example, fragmentation breakpoints can be used to identify the likely tissue of origin of a molecule in cell-free DNA. In oncology, this can be used to identify the specific type or subtype of cancer, particularly in screening tests.

In some embodiments, provided herein are methods comprising: contacting a sample with a probe that differs in complementarity to a target region of a first nucleic acid molecule relative to a second nucleic acid molecule in the sample; optionally activating probe hybridized to said first nucleic acid molecule by selectively modifying said probe hybridized to said first nucleic acid molecule relative to probe hybridized to said second nucleic acid molecule; selectively digesting probe hybridized to said first or said second nucleic acid molecule relative to the other; and sequencing at least a portion of probe that was hybridized to said first nucleic acid molecule. In some embodiments, the first and second nucleic acid molecules comprise end-repaired nucleic acid molecules. In some embodiments, the first and second nucleic acid molecules are A-tailed nucleic acid molecules. In some embodiments, the first and second nucleic acid molecules comprise an adapter sequence. In some embodiments, the first and second nucleic acid molecules are amplified.

In some embodiments, the probe is activated by contact with a cleavage agent. In some embodiments, the cleavage agent is selected from the group consisting of a restriction endonuclease, a flap endonuclease, a mismatch repair enzyme, an RNase, a Cas protein, an argonaute family enzyme, a DNA-formamidopyrimidine glycosylase (Fpg), an apurinic/apyrimidinic (AP) endonuclease (APE 1), and a chemical cleavage agent.

In some embodiments, the digesting comprises contacting the probe with an exonuclease or endonuclease. In some embodiments, the exonuclease is a 3′ to 5′ exonuclease. In some embodiments, the exonuclease is a 5′ to 3′ exonuclease. In some embodiments, the digesting comprises a pyrophosphorolysis reaction.

In some embodiments, the probe or the first and/or second nucleic acid molecules comprises a binding moiety at its 3′ end, 5′ end, or internally. In some embodiments, a binding moiety is added to a molecule via extension of the molecule in the presence of a nucleotide comprising a binding moiety. In some embodiments, the binding moiety is biotin.

In some embodiments, the probe or the first and/or second nucleic acid molecules comprises a linker that can be hybridized to a complementary sequence bound to a solid support.

In some embodiments, the method further comprises the step of capturing the probe or the first and/or second nucleic acid molecules on a surface prior to the digesting.

In some embodiments, the method further comprises the step of capturing the probe or the first and/or second nucleic acid molecules on a surface after the digesting.

In some embodiments, the surface comprises a bead.

In some embodiments, the method further comprises the step of differentially releasing the probe hybridized to the first nucleic acid molecule or the second nucleic acid molecule. In some embodiments, the releasing comprises increasing temperature. In some embodiments, the releasing comprises changing pH. In some embodiments, the releasing comprises changing salt concentration. In some embodiments, the releasing is caused by the digesting. In some embodiments, activation cleaves the probe and the probe melts from the fragment without requiring a change in reaction conditions.

In some embodiments, the method further comprises the step of detecting the first or second nucleic acid molecule. In some embodiments, the detecting comprises sequencing a probe molecule that was hybridized to the first or second nucleic acid molecule.

In some embodiments, the probe comprises a base that is complementary to a position in the first nucleic acid molecule and is mismatched with a corresponding position in the second nucleic acid molecule. In some embodiments, the digesting comprises contacting probe hybridized to said first and second nucleic acid molecules with an enzyme that preferentially digests complementary strands over non-complementary strands or with an enzyme that preferentially digests non-complementary strands over complementary strands.

In some embodiments, the probe comprises a 3′ end, 5′ end, or internal blocking group. In some embodiments, the probe is activated using a cleavage agent that removes the blocking group from probe hybridized to the first nucleic acid molecule but not from probe hybridized to the second nucleic acid molecule. In some embodiments, the digesting comprises contacting probe with a nuclease that cleaves probe lacking a blocking group but does not cleave probe having a blocking group.

Also provided herein are kits comprising reagents sufficient for, necessary for, or useful for conducting any of the methods above on a sample comprising the first and second nucleic acid molecules.

Further provided herein are methods comprising: contacting a sample with a probe that differentially hybridizes to a target region of a first nucleic acid molecule relative to a second nucleic acid molecule in the sample, wherein the first or second nucleic acid molecule comprises a gene fusion or indel; optionally activating probe hybridized to the first nucleic acid molecule by selectively modifying the probe hybridized to the first nucleic acid molecule relative to probe hybridized to the second nucleic acid molecule; selectively digesting probe hybridized to the first or said second nucleic acid molecule relative to the other; and detecting the first and/or second nucleic acid molecule.

In some embodiments, the probe differentially hybridizes to the first nucleic acid molecule relative to the second nucleic molecule based on differential complementarity of the probe to the first nucleic acid relative to the second nucleic acid.

In some embodiments, the probe differentially hybridizes to the first nucleic acid molecule relative to the second nucleic molecule based on the presence of a blocking oligonucleotide hybridized to the first nucleic acid molecule but not the second nucleic acid molecule, wherein a 3′ region of the probe is blocked by the blocking oligonucleotide. In some embodiments, the blocking oligonucleotide selectively hybridizes to wild-type sequence relative to sequence comprising a gene fusion or indel.

In some embodiments, the first or second nucleic acid molecule is enriched relative to the other following the digesting.

In some embodiments, the digesting comprises pyrophosphorolysis. In some embodiments, the digesting comprises contacting the sample with the probe, a pyrophosphorolysing enzyme, and a source of pyrophosphate ion.

In some embodiments, the probe, the first nucleic acid molecule, and/or the second nucleic acid molecule is attached to solid support. In some embodiments, the probe, the first nucleic acid molecule, and/or the second nucleic acid molecule is attached to the solid support prior to the digesting.

In some embodiments, the probe comprises a 5′ tail region that is not complementary to the first or second nucleic acid molecules. In some embodiments, the 5′ tail region is complementary to a capture oligonucleotide attached to a solid support.

In some embodiments, the probe, the first nucleic acid molecule, and/or the second nucleic acid molecule comprises a capture moiety. In some embodiments, the capture moiety is biotin and the solid support comprises streptavidin. In some embodiments, the solid support is a bead. In some embodiments, the bead is a magnetic or paramagnetic bead.

In some embodiments, the one or more wash steps are performed between one or more of the steps described above.

In some embodiments, the sample is an adaptor tagged library of nucleic acid molecules.

In some embodiments, the detecting comprises a nucleic acid amplification step.

In some embodiments, the detecting comprises sequencing. In some embodiments, the sequencing comprises Next Generation Sequencing (NGS).

In some embodiments, multiple different probe oligonucleotides are employed, each with a different sequence designed to anneal to a different target sequence.

In some embodiments, the sample is, or is derived from, a human blood or tissue sample.

In some embodiments, the probe comprises a ‘5 region complementary to a portion of a wild-type sequence and a 3’ portion not complementary to the wild-type sequence.

Also provided herein are kits comprising reagents sufficient, necessary, or useful for conducting the method described above on a sample comprising said first and second nucleic acid molecules. In some embodiments, the kits comprise one or more or all of: the probe, an activation reagent, a digestion reagent, a solid support, a buffer, a capture reagent, an amplification reagents, a sequencing reagent, a positive control, a negative control, a sequencing adapter, a ligase, a library preparation reagent, a crowding agent, and a detergent.

### BRIEF DESCRIPTION OF THE DRAWINGS

FIG. 1 shows an exemplary mutation specific methodology. Adapter sequences labelled X and Y′ are attached to fragmented nucleic acid. The sequence is amplified with biotin (B) labelled primers. The amplicon is bound to a streptavidin bead and contacted with a probe having a 5′ P5 sequencing adapter sequence and a multiplex identifier (MID) sequence. The probe differentially hybridizes to mutant and wild-type sequences (position N identifying the location of a sequence difference). Probes are exposed to phosphorolysis conditions generating a PPL'ed short probe and a non-PPLed probe. P7′ adapters are ligated to the 3′ end of the probes and the resulting products are amplified and sequenced.

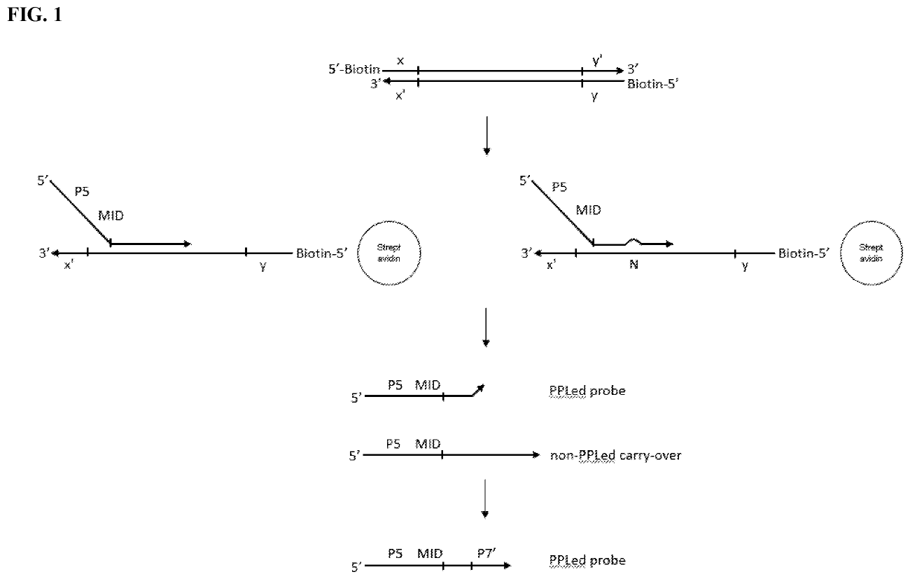

FIG. 2 shows an exemplary mutation specific methodology. Adapter sequences labelled X and Y′ are attached to fragmented nucleic acid. The sequence is amplified. The amplicon is contacted with a probe having a 5′ P5 sequencing adapter sequence and a multiplex identifier (MID) sequence and exposed to phosphorolysis conditions generating a PPL'ed short probe and a non-PPLed probe. A bead-bound capture oligonucleotide captures non-PPL'ed probe, but not PPL'ed probe. The uncaptured probe is isolated and optionally amplified and sequenced.

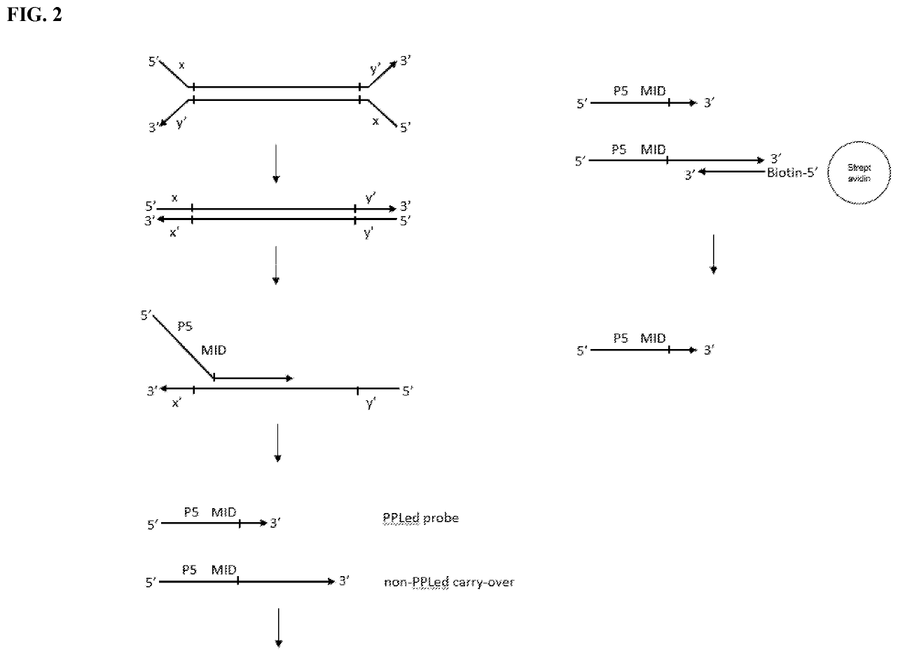

FIG. 3 shows an exemplary mutation-agnostic methodology. P7 adapter sequences, stem sequences, and a biotin moiety are attached to fragmented nucleic acid. The products are captured on streptavidin beads and contacted with a probe comprising a 5′ P5 and MID sequence. Probes are extended and the products are amplified and sequenced.

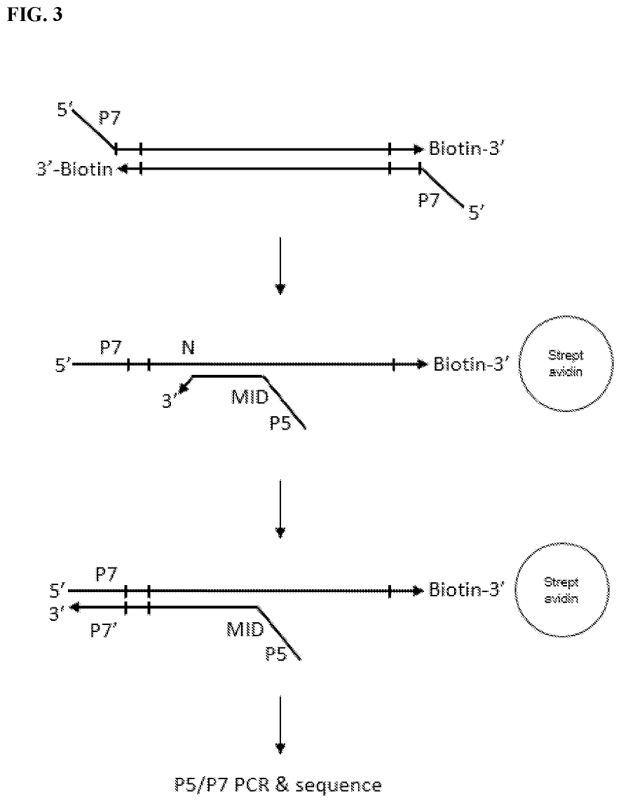

FIG. 4A shows an exemplary mutation-agnostic methodology. P7′ adapter sequences, stem sequences, and a biotin moiety are attached to fragmented double stranded nucleic acid. The products are denatured and captured on streptavidin beads and contacted with a probe having a non-complementary 5′ flap. Probes are extended and the products are amplified and sequenced.

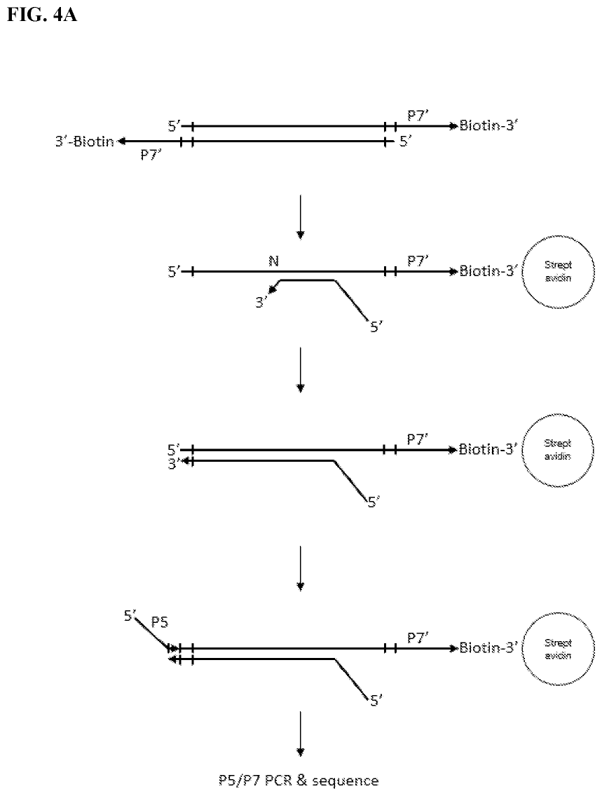

FIG. 4B shows an exemplary mutation-agnostic methodology. P7′ adapter sequences, stem sequences, and a biotin moiety are attached to fragmented nucleic acid. The products are captured on streptavidin beads and contacted with a probe having a 5′ flap with a P5 adapter sequence (middle panel) and alternatively connected to the captured fragment by hairpin loop (lower panel). Following cleavage, probes are amplified and sequenced.

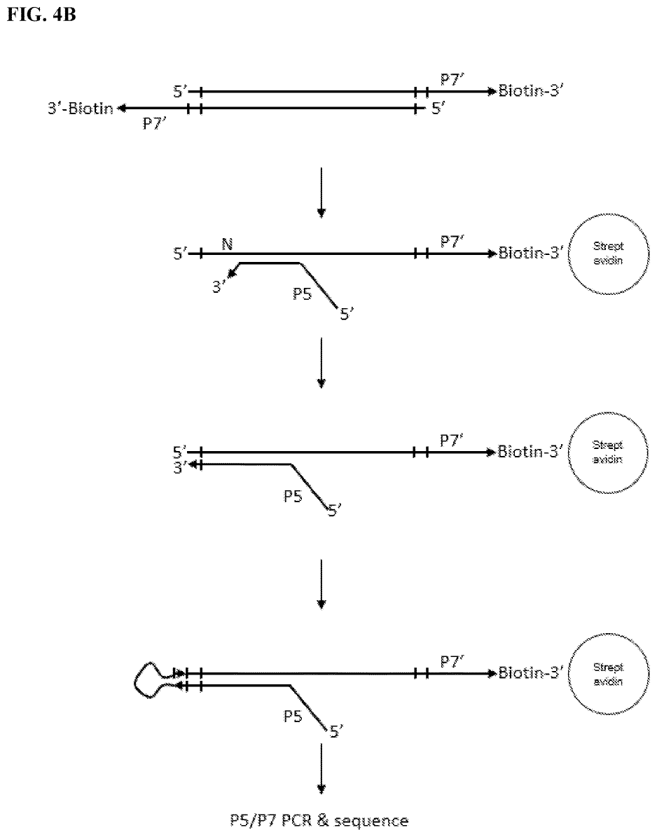

FIG. 5 shows an exemplary mutation-agnostic methodology. P7 adapter sequences and stem sequences are attached to fragmented double stranded nucleic acid. The products are denatured and contacted with probes comprising a 5′ P5 adapter sequence. Probes are extended using biotin-labelled nucleotides and then captured on streptavidin beads. Following cleavage, probes are amplified and sequenced.

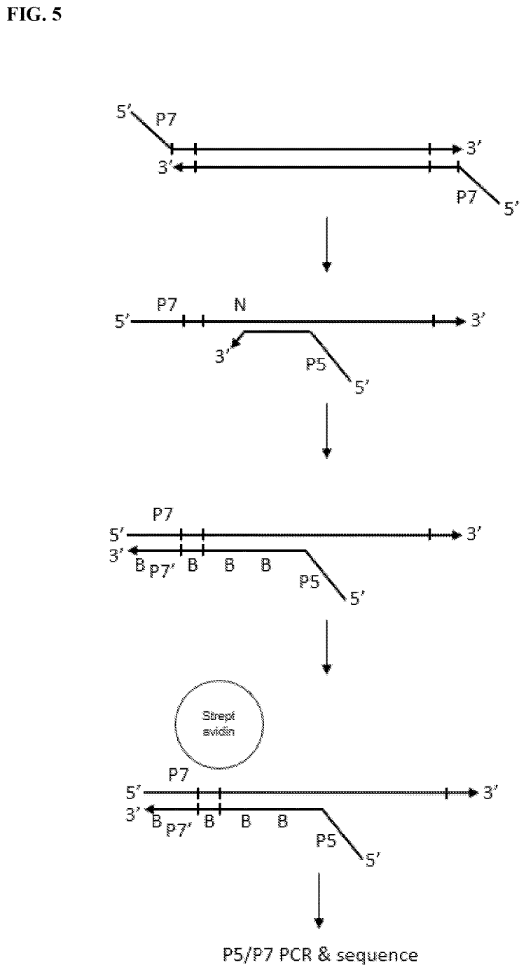

FIG. 6 shows an exemplary methodology for detection of gene fusions.

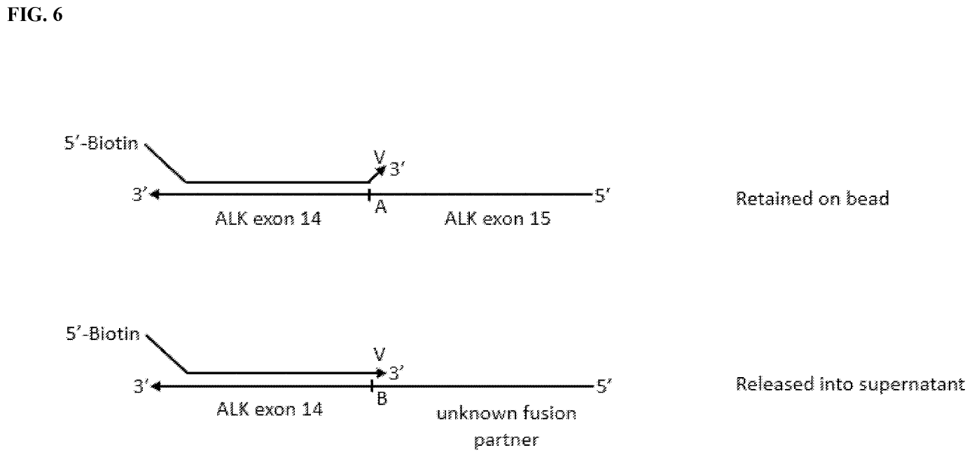

FIG. 7 shows an exemplary methodology for detection of gene fusions.

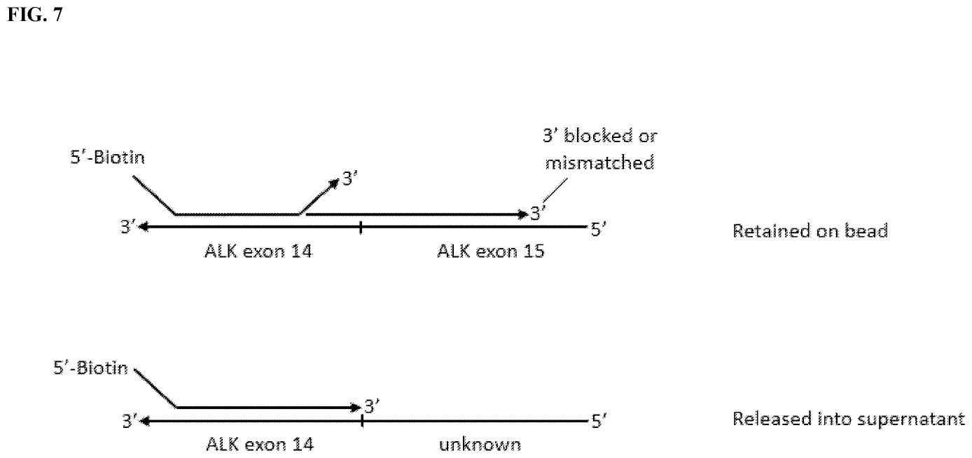

FIG. 8 shows an assay design for the experimental work described in Example 1.

FIG. 9 shows an assay design using target nucleic acid molecules that have undergone library preparation.

FIG. 10 shows a gel analysis of differentially processed probe between wild-type and mutant (BRAF V600E mutation) target nucleic acid sequences.

FIG. 11 shows a mutation agnostic assay design.

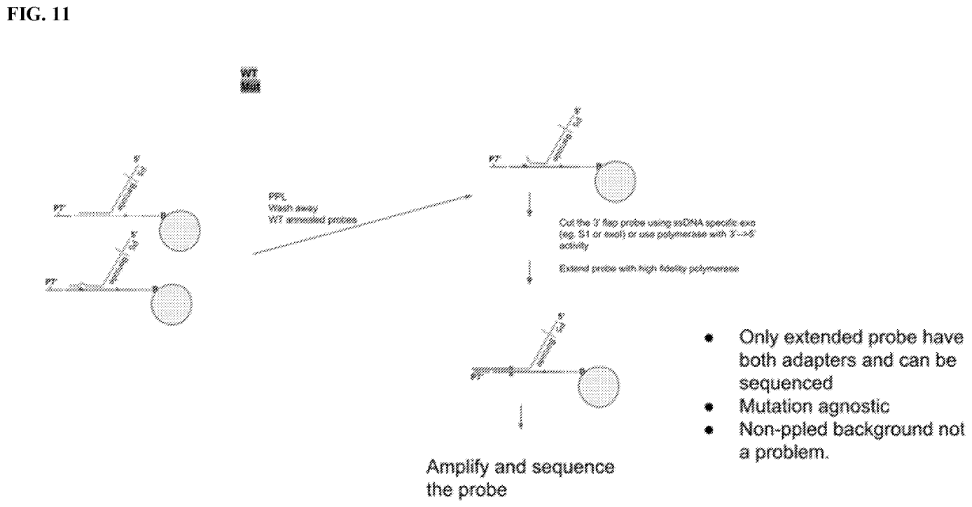

FIG. 12 shows a mutation agnostic assay design using target nucleic acid molecules that have undergone library preparation.

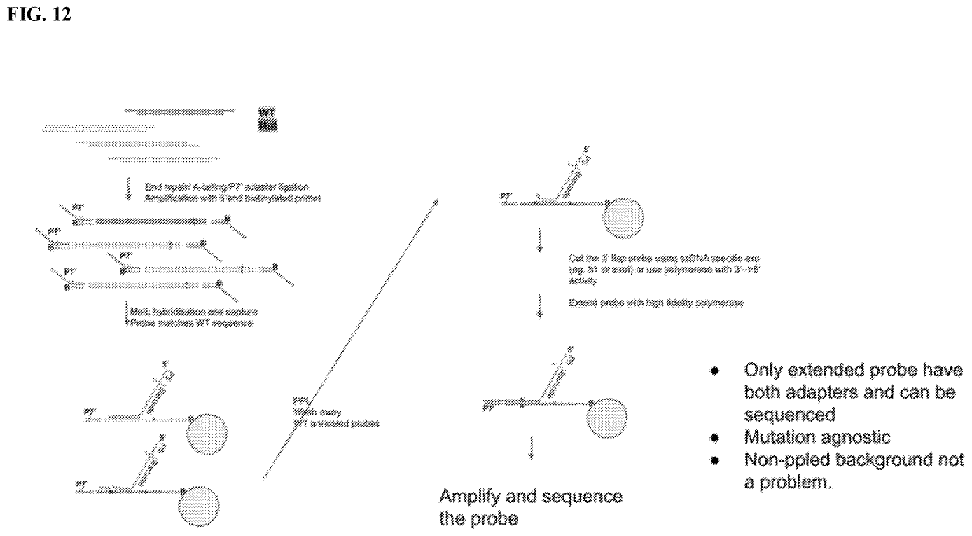

### DETAILED DESCRIPTION OF THE INVENTION

In some embodiments, the systems and methods employ one or more probes that hybridize, at least partially, to desired and/or undesired sequences. The probes are configured such that they are differentially cleaved or degraded in the presence of the desired sequence(s) relative to the undesired sequence(s). As used herein, the term “target molecules” refers to the nucleic acids molecules in a sample that hybridize to the probe. In some embodiments, probes hybridize to target molecules at either their 3′ end or 5′ end, or internally. In some embodiments, either the probes or the target molecules are attached to a solid surface. In some embodiments, probes comprise DNA. In some embodiments, probes comprise RNA. In some embodiments, probes comprise a mixture of DNA and RNA. In some embodiments, probes comprise one or more synthetic bases (e.g., locked nucleic acid (LNA)). In some embodiments, probes comprise a synthetic backbone modification.

In some embodiments, the systems and methods cleave or digest the probe in a probe/target hybridization complex. In some embodiments, the cleavage/digestion mechanism employed is selected to one strand (e.g., the probe). In some embodiments, the cleavage/digestion mechanism is not strand-selective. In some embodiments, the probe or target is protected through modification, for example, of primers or adaptors used to create the modified nucleic acid (e.g., 3′/5′ end-blocking modifications, internal base/backbone modifications, etc.). In some embodiments, the probe or target is protected through the introduction of modification during amplification (e.g., phosphorothioate modification of the backbone).

In some embodiments, an activation step is employed to prepare probes for a cleave/digestion reaction. In some embodiments, probes are rendered undigestible via a 3′, 5′, or internal modification. In some embodiments, the modification is a mismatch relative to the target. In some embodiments, the mismatch is at an end (e.g., 3′ end or 5′ end) of the probe. In some embodiments, the mismatch is internal. In some embodiments, the mismatch comprises two or more bases. In some embodiments, the mismatch region provides a flap or bubble when the probe is hybridized to a target. In some embodiments, the probe is prepared with one or more modifications (e.g., backbone modifications such at phosphorothioate (PTO), base modifications, linkers etc). In some embodiments, the modification is inclusion of RNA bases in an otherwise DNA probe. In some embodiments, the modification comprises circularization of the probe.

In some embodiments, such probes are selectively ‘activated’ based on their differential hybridization to different targets (e.g., alleles), allowing the probes to be subsequently digested. In some embodiments, the activation step provides a nick or gap in the probe.

In some embodiments, activation occurs using one or more restriction endonucleases. In some embodiments, the restriction endonuclease is a methylation/modification-specific endonuclease. In some embodiments, activation occurs using a Flap endonuclease. In some embodiments, the activation occurs using a mismatch repair enzyme or mismatch-specific endonuclease (e.g., CelI, S1, T7E1, SURVEYOR nuclease). In some embodiments, activation occurs using an RNase enzyme (e.g., RNaseH2). In some embodiments, activation occurs using a CRISPR/Cas systems (e.g., including use of Cas enzymes or mutants that cleave only one strand). In some embodiments, activation occurs using an argonaute family enzyme (e.g., Ago1, Ago2, Ago3, Ago4, Hili, Hiwi, Hiwi2, Hiwi3, aubergine, PIWI, Ago5, Ago6, Ago7, Ago8, Ago9, Ago10, Alg-1, Alg-2, etc.). In some embodiments, activation occurs using chemical cleavage of mismatch (CCM) (e.g., hydroxylamine+potassium permanganate followed by piperidine).

In some embodiments, digestion of the probe is selective based on differential hybridization to the target. In some embodiments, digestion of the probe employs a general digestion method that is selective for activated probes. In some embodiments, digestion is 3′-5′ in direction. In some embodiments, digestion is 5′-3′ in direction. In some embodiments, digestion is via internal cleavage of the probe.

In some embodiments where 3′-5′ digestion is desired, a 3′-5′ exonuclease is employed. Such exonucleases include, but are not limited to, Exonuclease I (ExoI) (e.g., E. coli ExoI), Exonuclease T (ExoT), Exonuclease VII (ExoVII), Exonuclease III (ExoIII), and DNS.

In some embodiments where 3′-5′ digestion is desired, a polymerase having 3′-5′ exonuclease activity is employed (e.g., proofreading polymerases, e.g., PHUSION (Thermo Fisher), Q5 (New England Biolabs), KAPA HIFI (Roche), KOD DNA polymerase).

In some embodiments where 5′-3′ digestion is desired, a 5′-3′ exonuclease is employed (e.g., Lambda Exonuclease, RecJf, T7 exonuclease, Exonuclease V (ExoV), Exonuclease VIII (ExoVIII), T5 exonuclease).

In some embodiments where 5′-3′ digestion is desired, a DNA polymerase having 5′-3′ exonuclease activity is employed. In some such embodiments, a 5′ (or internally) blocked probe can act as a blocking oligonucleotide, preventing extension of a primer against the target. Selective cleavage of the probe based on differential hybridisation results in melting of the blocked 5′ end, enabling extension of an upstream primer, during which the remaining hybridised probe is digested in the 5′-3′ direction (or displaced via strand displacement), releasing the target molecule. In a similar embodiment, the 5′ section of the cleaved probe could itself act as the primer.

For internal cleavage, any of the “activation” methods described above can be used as a standalone digestion method. In some embodiments, the resulting fragments from the cleaved probe have a lower melting temperature (Tₘ) than the intact probe, allowing selective denaturation from the target by an increase in temperature, pH etc. In some embodiments, the activation step itself is sufficient to permit a probe to melt from the fragment without requiring reaction condition changes.

In some embodiments, the present systems and methods use selective digestion as way of isolating, enriching for, or depleting nucleic acid sequences to facilitate subsequent detection (e.g., via sequencing). In some embodiments, the digestion reaction relies on complementarity between hybridised strands, and only digests strands having a particular characteristic (e.g., a specific sequence or structure). The reaction selectively shortens or digests certain sequences having the particular characteristic, while leaving sequences not having the characteristic undigested or less digested. The reaction can be performed such that molecules hybridized to the shortened or more digested sequences are recovered and analysed or such that the shortened or more digested probes themselves are recovered and analysed. Alternatively, the reaction can be performed such that less shortened or more digested sequences are analysed. Alternatively, the reaction can be performed so that the sequences which have not undergone any digestion are analysed.

The reactions may be repeated one or more times to further enrich or isolate a sample for the sequence of interest. For example, in some embodiments, a second round of enrichment, depletion, or isolation occurs after the first round is completed using the same reagents as the first round. In some embodiments, an amplification reaction can be used between a first and second round of enrichment.

In some embodiments, the separation of any probe complexes that were better (e.g., perfectly) annealed occurs by using reaction conditions that favour probe sequence complexes which were better (e.g., perfectly) annealed over probe sequence complexes with less (e.g., imperfect) annealing. This could take the form of changes to the reaction mixture temperature and/or changes to the pH of the reaction mixture and/or changes to the salinity of the reaction mixture. In some embodiments, chemical agents (e.g., dimethylsulfoxide (DMSO), formamide, etc.) are employed to denature nucleic acids to facilitate enrichment or depletion. In some embodiments, nucleic acid molecules (displacing oligonucleotides) and enzymes or proteins are used separate hybridized nucleic acid molecules. In some embodiments, cleavage of the nucleic acid causes the oligonucleotide to melt off without requiring a change in reaction conditions.

In some embodiments, two probes are employed, one for a forward strand of a double-stranded target nucleic acid and one for the reverse stand. In some embodiments, it is beneficial to design the probes so that they do not hybridise to each other in a manner that would interfere with the desired reaction.

In some embodiments, probes or targets are captured onto a solid support prior to, or following, exposure of probes to samples comprising targets. In some embodiments, probes or targets comprise a biotin moiety that is captured by a corresponding streptavidin moiety on a solid support. In some embodiments, probes or targets are hybridised to linker molecules which themselves comprise a modification, for example a biotin moiety, through which they are captured on a solid support. In some embodiments, the solid support is a bead. In some embodiments, the bead is a magnetic or paramagnetic bead.

In some embodiments, one or more wash steps are performed between one or more of the steps. In some embodiments, wash and hybridisation steps are performed at elevated temperatures between 25-95° C.

In some embodiments, the sample comprises an adaptor tagged library of nucleic acid analytes.

In some embodiments, the sequences are identified by an amplification reaction (e.g., polymerase chain reaction (PCR), nucleic acid sequence-based amplification (NASBA), multiple displacement amplification (MDA), transcription-mediated amplification (TMA), strand displacement amplification (SDA), rolling circle amplification (RCA), loop mediated isothermal amplification (LAMP), recombinase polymerase amplification (RPA), helicase dependent amplification (HDA), nicking and extension amplification reaction (NEAR), and the like). In some embodiments, the sequences are identified by microarray analysis.

In some embodiments, the sequences are identified by sequencing. In some embodiments, the sequences are identified by Next Generation Sequencing (NGS) (e.g., bridge amplification sequencing (Illumina), SMRT sequencing (PacBio), Ion Torrent sequencing, nanopore sequencing, pyrosequencing, and the like).

The compositions and methods of the invention may be employed against any type of sample, including, but not limited to environmental (e.g., water, soil, air, etc.) samples and biological samples. Biological samples may be from any source including plants, animals, infectious disease agents, and the like. Suitably, in some embodiments, the analytes/sequences are derived from a biological sample taken from a mammalian subject (especially a human patient) such as blood, plasma, sputum, urine, skin, biopsy or surgical resection. In some embodiments, the biological sample will be subjected to lysis in order that the analytes/sequences are released by disrupting any cells present. In other embodiments, the analytes/sequences may already be present in free form within the sample itself; for example cell-free DNA circulating in blood or plasma. The compositions and methods of the invention find particular use with historically challenging sample types that may have low allele fractions of the analyte of interest. Such samples include blood, urine, cytosponge-collected samples (e.g., oesophageal samples), bronchoalveolar lavage (BAL) derived samples, pleural fluid, and cerebrospinal fluid (CSF).

In some embodiments, samples are pooled samples. Pooled samples involve mixing multiple samples togethers in a batch where the pooled collection is tested. This approach increases the number of individual samples that can be tested using a more limited amount of resources. Pooled samples of interest include, but are not limited to, donated blood samples, agricultural samples, food samples, sperm samples, and biological samples tested for the presence of infectious disease agents (e.g., SARS-CoV-2, HIV, HCV, etc.). In some embodiments, the pooled sample is an environmentally collected sample (e.g., wastewater sample) that has, by the nature of its generation, pooled samples from multiple different sources. While pooling of samples may reduce the allele fraction of variants as the samples dilute each other, it can provide a dramatic increase in efficiency of screening. Because the technology provided herein enables detection at very low allele fractions, it is particularly well suited for analysis of pooled samples. In some embodiments, a fraction of each initial sample is pooled without use of barcodes or other complex preparation steps and the pooled sample is tested. If a positive result is obtained, remaining fractions of the unpooled samples may be tested individually.

The target nucleic analysed may be any target nucleic acid of interest. In some embodiments, the analysis is for research, diagnostic, or therapeutic purposes. In some embodiments, where the samples are from human subjects, the purpose may be for the analysis of, detection of, treatment of, or selection of treatment of one or more diseases or conditions. The technology finds particular use for the analysis of multifactorial diseases and disorders, including, but not limited to, cancers (e.g., bladder, breast, cervical, colorectal, ovarian, uterine, vaginal, vulvar, head and neck, eye, brain, kidney, liver, lung, lymphoma, mesothelioma, myeloma, prostate, skin, thyroid, pancreatic, bone, esophageal, gallbladder, stomach, testicular, anal, rectal, oral, salivary gland, sarcoma, and thyroid), autoimmune diseases, asthma, ciliopathies, cleft palate, diabetes, heart disease, hypertension, inflammatory bowel disease, intellectual disabilities, mood disorders, obesity, infertility, and refractive error.

In some embodiments, the nucleic acid analysed is a methylated sequence or is derived from a methylated sequence. The technology is used to differentiate methylation status at any particular or multiple locations in a target nucleic acid. Methylated sequences may first be modified using chemical treatments (e.g., oxidation, reduction, bisulfite treatment) or by exposure to methylation dependent restriction enzymes or any other suitable approach followed by enrichment and/or identification of the modified sequence.

In some embodiments, the nucleic acid analysed has a modified base, for example 8-oxoguanine, which may be a consequence of DNA damage.

In some embodiments, targeted regions of RNA present in the sample are transcribed into DNA.

Some embodiments of the technology employ a solid surface. Any solid surface compatible with nucleic acid capture, directly or indirectly, may be employed. Solid supports include, but are not limited to materials made of, glass, metals, gels, plastics (e.g., polystyrene), ceramics, and filter paper, among others. In some embodiments, the solid support is a bead (e.g., a paramagnetic bead), microparticle, nanoparticle, column, slide, or the like. In some embodiments, the solid support is modified to include one or more of the following functional groups: amine, carboxylate, sulfonate, trimethylamine and/or epoxide to facilitate reactions with or coupling or conjugation to biomolecules. In some embodiments, Streptavidin is attached to the solid surface. In some embodiments, the solid support is a dextran-modified surface. In some embodiments, the solid support is a Polyethylene Glycol (PEG) or PEG-modified surface. In some embodiments, the solid support is a Polyvinylpyrrolidone (PVP) or PVP-modified surface. In some embodiments, the solid support is a polysaccharide or polysaccharide-modified surface. In some embodiments, the polysaccharide is selected from one or more of dextran, ficoll, glycogen, gum arabic, xanthan gum, carageenan, amylose, agar, amylopectin, xylans and/or beta-glucans. In some embodiments, the solid support is a chemical resin or chemical resin-modified surface. In some embodiments, the chemical resin or chemical-resin modified surface is selected from one or more of the following resins: isocyanate, glycerol, piperidino-methyl, polyDMAP (polymer-bound dimethyl 4-aminopyridine), DIPAM (Diisopropylaminomethyl, aminomethyl, polystyrene aldehyde, tris(2-aminomethyl) amine, morpholino-methyl, BOBA (3-Benzyloxybenzaldehyde), triphenyl-phosphine or benzylthio-methyl.

In some embodiments, a capture moiety is used to associate a probe or target nucleic acid with a solid surface. In some embodiments, a capture moiety is covalently attached to the solid support via a chemically-cleavable linker, such as a disulfide, allyl, or azide-masked hemiaminal ether linker. In some embodiments, the capture moiety is covalently attached to the solid support via amide or phosphorothioate bonds. In some embodiments, the probe or target may incorporate a moiety that is modified to introduce a capture moiety. In some embodiments, the reaction includes reacting an alkyne labelled oligonucleotide with an azide biotin conjugate.

In some embodiments, the capture moiety comprises an oligonucleotide sequence and the solid surface comprises a complementary oligonucleotide sequence. In some embodiments, the oligonucleotide sequence comprises one or more modified bases and/or other such modifications known to the person skilled in the art, to change the melting temperature. In some embodiments, the presence of one or more modified bases and/or other such modifications known to the person skilled in the art leads to a decrease in the melting temperature. In some embodiments, the presence of one or more modified bases and/or other such modifications known to the person skilled in the art leads to an increase in the melting temperature. In some embodiments, the length of the complementary sequence is between 10, 20, 30, 40, 50, 100, 150 and 200 bases. In some embodiments, the length of the complementary sequence is between 10, 20, 30, 40, 50, and 100 bases. In some embodiments, the length of the complementary sequence is between 10-20, 10-30, 10-40 and 10-50 bases. In some embodiments, the length of the complementary sequence is between 10-20, 10-30 and 10-40 bases. In some embodiments, the length of the complementary sequence is between 10-20 and 10-30 bases. In some embodiments, the length of the complementary sequence is between 10-20 bases.

In some embodiments, the capture moiety comprises a chemical modification and is attached to the solid support via an interaction between the chemical modification and the solid support. In some embodiments, the chemical modification is biotin and the solid support further comprises streptavidin. In some embodiments, captured oligonucleotide sequences are released from the solid support. In some embodiments, captured oligonucleotide sequences are released from the solid support by chemical denaturation. In some embodiments, chemical denaturation is achieved by the use of suitable concentration of base. In some embodiments, 0.1M of NaOH may be used. In some embodiments, oligonucleotide sequences are released from the solid support by the cleavage of a chemical linker through the addition of tris(2-carboxyethyl) phosphine (TCEP) or dithiothreitol (DTT) for a disulfide linker; palladium complexes or an allyl linker; or TCEP for an azide-masked hemiaminal ether linker. In some embodiments, oligonucleotide sequences are released from the solid support by removing a non-canonical base, from oligonucleotide sequences, and cleavage at the resultant abasic site. In some embodiments, the non-canonical base is uracil, which is removed by uracil DNA glycosylase. In an alternate embodiment, the non-canonical base is 8-oxoguanine, which is removed by formamidopyrimidine DNA glycosylase (Fpg).

In some embodiments, the capture moiety is an oligonucleotide region and release is performed through heating of the reaction mixture. In some embodiments, the reaction mixture is heated to 37° C.-100° C. over 1-20 minutes. In some embodiments, the reaction mixture is heated over 1-15 minutes. In some embodiments, the reaction mixture is heated over 1-10 minutes. In some embodiments, the reaction mixture is heated over 1-5 minutes. In some embodiments, the reaction mixture is heated over 5 minutes. In some embodiments, the reaction mixture is heated to 37° C.-85° C. In some embodiments, the reaction mixture is heated to 37° C.-75° C. In some embodiments, the reaction mixture is heated to 37° C.-65° C. In some embodiments, the reaction mixture is heated to 37° C.-55° C. In some embodiments, the reaction mixture is heated to 37° C.-45° C.

The person skilled in the art will appreciate that the temperature to which the reaction mixture is heated to enact release of complementary oligonucleotide regions depends on a number of factors including the length of the regions.

In some embodiments, release is achieved through the cleavage of one or more oligonucleotide sequences. This cleavage can be achieved by any of the means previously, or subsequently, described or by any of the means known to the person skilled in the art. In some embodiments, oligonucleotide sequences are cleaved chemically. In some embodiments, oligonucleotide sequences are cleaved enzymatically. In some embodiments, oligonucleotide sequences are cleaved by a restriction enzyme. In some embodiments, oligonucleotide sequences are cleaved by epigenetic modification sensitive or dependent restriction enzymes. In some embodiments, oligonucleotide sequences are cleaved by methylation sensitive or dependent restriction enzymes. In some embodiments, oligonucleotide sequences are cleaved by hydroxymethylation sensitive or dependent restriction enzymes.

In some embodiments, prior to, or after, cleavage/digestion of variant or wild-type sequences or probes hybridized thereto, sequences are enzymatically or chemically converted to allow detection of their methylation status.

In some embodiments, oligonucleotide sequences comprise a photocleavable linker and oligonucleotide sequences are released from the solid support by cleavage of this linker (e.g., by UV light). This modification may be a chemical backbone modification.

In some embodiments the sample nucleic acids are fragmented prior to application of the methods disclosed herein. The person skilled in the art will appreciate that there are multiple techniques which may be used to fragment DNA. Such methods include sonication, needle shear, nebulisation, point-sink shearing, passage through a pressure cell (French press) and enzymatic methods. In some embodiments, fragmentation is achieved by sonication. In some embodiments, The Bioruptor® (Denville, NJ) device may be used. In some embodiments, fragmentation is achieved by acoustic shearing. In some embodiments, the Covaris® instrument (Woburn, MA) may be used. In some embodiments, fragmentation is achieved by nebulisation. Nebulization forces DNA through a small hole in a nebulizer unit, which results in the formation of a fine mist that is collected. Fragment size is determined by the pressure of the gas used to push the DNA through the nebulizer, the speed at which the DNA solution passes through the hole, the viscosity of the solution, and the temperature. In some embodiments, fragmentation is achieved by hydrodynamic shear. In some embodiments, The Hydroshear from Digilab (Marlborough, MA) may be used. In some embodiments, fragmentation is achieved by point-sink shearing. In some embodiments, fragmentation is achieved by needle shearing. In some embodiments, fragmentation is achieved via use of a French press. In some embodiments, fragmentation is achieved by enzymatic fragmentation. In some embodiments, fragmentation is achieved by restriction endonuclease digestion. In some embodiments, fragmentation is transposome mediated fragmentation. In some embodiments, fragmentation is achieved by Cas9. In some embodiments, fragmentation is achieved by Cas9, as described in U.S. Pat. No. 10,577,644, herein incorporated by reference in its entirety. In some embodiments, one or more different fragmentation techniques may be used.

In some embodiments, one or more of the same or different fragmentation techniques may be used at one or more different points of the method.

In some embodiments, fragmentation and adaptor tagging of sequences occurs at the same time or in the same step of the method. One such example is Nextera DNA Library Prep Kit by Illumina.

The person skilled in the art will appreciate that there are multiple techniques which may be used to prepare adaptor tagged sequences/libraries.

In some embodiments, following fragmentation, the ends of nucleic acids may be polished and A-tailed prior to ligation to one or more adaptors.

In some embodiments, following fragmentation, the ends of nucleic acids may be polished and ligated to adaptors in a blunt-end ligation reaction.

In some embodiments, following fragmentation, adaptors are ligated to single-stranded DNA.

In some embodiments, following fragmentation, a terminal transferase enzyme is used to add non-templated bases to the 3′ end of fragments, providing a site for priming to make fragments double-stranded.

In some embodiments, topoisomerase may be used in lieu of a DNA ligase.

In some embodiments, TOPO cloning may be used to add adaptors to fragmented DNA.

In some embodiments, following fragmentation, transposases can be used to add adaptor sequences to nucleic acids.

In some embodiments, following fragmentation, standard transposons can be used but then modified to create a Y-shaped adaptor using oligonucleotide replacement.

In some embodiments, wherein the sample is an adaptor tagged library, blocking oligonucleotides are used to prevent cross-hybridisation of library molecules (so called ‘daisy chaining’).

In some embodiments, the sample comprises one or more blocking oligonucleotides.

Any sequencing methodology may be used to analyse target nucleic acid or probe molecules. In some embodiments, the sequencing is Maxam-Gilbert sequencing. In some embodiments, the sequencing is Sanger sequencing. In some embodiments, the sequencing is shotgun sequencing. In some embodiments, the sequencing is single-molecule real-time sequencing. In some embodiments, the sequencing is ion semiconductor sequencing. In some embodiments, the sequencing is pyrosequencing. In some embodiments, the sequencing is sequencing by synthesis. In some embodiments, the sequencing is combinatorial probe anchor synthesis (cPAS). In some embodiments, the sequencing is sequencing by ligation. In some embodiments, the sequencing is nanopore sequencing. In some embodiments, the sequencing is GenapSys sequencing. In some embodiments, the sequencing is Next Generation Sequencing (NGS).

In some embodiments, there is provided a method for screening a patient comprising the use of any previously or subsequently described embodiment of the method to detect the presence or absence of one or more specific nucleic acid sequences in a sample derived from a patient.

The person skilled in the art will appreciate that such a screening will be useful for monitoring a patient receiving treatment for one or more conditions, the treatment status of which can be ascertained by the levels of one or more nucleic acid sequences in a patient sample.

For example, the treatment status of patients receiving treatment for one or more cancers can be ascertained by the levels of one or more nucleic acid sequences in their blood and/or the presence and/or absence of one or more specific variants. A high level of circulating tumour nucleic acid sequences and/or the presence and/or absence of one or more specific variants, can be used to deduce whether or not a particular treatment is having the desired effect. There is thus provided a method for monitoring the success, or not, of a particular treatment wherein such success can be inferred by the presence or absence of specific nucleic acid sequences and/or their respective levels in a sample derived from a patient.

In some embodiments, there is provided a method of monitoring a patient in remission to detect any recurrence of disease.

In some embodiments, there is provided a method of screen nominally healthy people to detect the presence of one or more disease states including, but not limited to, cancer.

In some embodiments, there is provided a method of detecting the presence and/or absence of one or more genetic markers in a patient diagnosed with one or more disease states and using the presence and/or absence of one or more markers to determine which treatment they should receive.

In some embodiments, there is provided a method for the diagnosis and/or monitoring of one or more cancers in a patient comprising the use of any previously or subsequently described embodiment of the method to detect the presence or absence of one or more specific nucleic acid sequences in a sample derived from a patient.

The person skilled in the art will appreciate that the one or more specific nucleic acid sequences may be specific to an individual (identified from a tissue biopsy or surgical resection, for example, by a method of identification such as sequencing) and that in such cases an individual patient specific panel may be used.

The person skilled in the art will further appreciate that in some embodiments, a panel will cover known hotspot regions of the human genome, those that are recurrently mutated in a given cancer type.

The person skilled in the art will further appreciate that in some embodiments a panel will cover a target region or the entirety of the human exome.

In some embodiments, there is provided a method for non-invasive prenatal testing (NIPT) comprising the use of any previously or subsequently described embodiments of the method to detect the presence or absence of one or more specific nucleic acid sequences in a sample derived from a patient, wherein the patient is a pregnant patient. In some embodiments, the sample is the plasma and or serum of the blood of a pregnant patient. In some embodiments, methods provided herein are employed to detect, enrich, and/or quantify a fetal fraction of a sample using a panel of common SNPs associated with such samples.

In some embodiments, there is provided a method of treating a patient comprising the steps of:
Performing any of the previously or subsequently described embodiments of the invention to detect the absence or presence of one or more specific nucleic acid sequences in a sample derived from a patient;
Making one or more treatment decisions based on the presence or absence of said sequences.

In some embodiments, the treatment decision is the initiation of a particular treatment. In some embodiments, the treatment decision is the cessation of a particular treatment. In some embodiments, the treatment decision is an increase in the dose of a particular treatment. In some embodiments, the treatment decision is a decrease in the dose of a particular treatment. In some embodiments, the treatment decision is an increase in the frequency of administration of a particular treatment. In some embodiments, the treatment decision is a decrease in the frequency of administration of a particular treatment. In some embodiments, the treatment decision is the addition of an additional drug to an existing treatment regimen. In some embodiments, the treatment decision is the removal of a drug from an existing treatment regimen.

In some embodiments, kits are provided that contain one or more or all of the components necessary, sufficient, or useful for carrying out a method as described herein. For example, in some embodiments, the kit comprises one or more probes, solid supports, enzymes, blocking oligonucleotides, buffers, detergents (e.g., sodium dodecyl sulfate (SDS), TWEEN20, etc.), adapters, crowding agents (e.g., polyethylene glycol (PEG), poly(vinyl alcohol) (PVA), dextran sulphate, etc.), solvents (e.g., formamide, ethylene carbonate, etc.), additives that hybridize to repetitive sequences (COT-1 DNA, salmon sperm DNA, oligonucleotides that block ribosomal RNA, and the like), capture moieties (e.g., biotin; e.g., as part of a probe), metal ions, blocking oligonucleotides, sequencing reagents, amplification reagents (isothermal amplification reagents; exponential amplification reagents (e.g., thermostable polymerase, primers, dNTPs, buffers, labelled detection probes)), transcription reagents, instructions for use, software, instruments, positive controls, negative controls, and the like. One or more containers may separately house one or more of the components.

In some embodiments, there is provided a kit comprising multiple probes, as previously or subsequently described.

In some embodiments, there is provided a kit comprising 1-1,000,000 individual probes. In some embodiments, there is provided a kit comprising 1-100,000 individual probes. In some embodiments, there is provided a kit comprising 1-10,000 individual probes. In some embodiments, there is provided a kit comprising 1-1,000 individual probes.

In some embodiments, bioinformatics approaches are used to analyse sequencing data. In some embodiments, the presence or absence of specific variants is called. In other embodiments, data from multiple variants are combined to derive a probabilistic estimate for the presence or absence of specific target nucleic acid or disease state.

For example, Illumina sequencing data analysis includes conversion and demultiplexing of BCL files into FASTQ format using tools such as bcl2fastq. In some embodiments, sequencing reads include molecular identifiers. In this case, molecular identifiers can be extracted from sequencing reads, appended to FASTQ headers, and the sequencing reads clipped. In some embodiments, barcodes with non-canonical bases (not A, C, G or T) can be filtered. The resulting reads can then be aligned using a tool such as bwa mem, using the —C option to append barcode sequences to alignments. Alignments can then be sorted by coordinate, duplicate reads marked, and reads annotated with read coordinate, mate coordinate and optical duplicate auxiliary tags using biobambam2, bamsormadup, and bammarkduplicatesopt. Reads can be filtered if they are not marked as proper-pairs or were marked as optical duplicate, supplementary, QC fail, unmapped or secondary alignments. Each read can then be marked with an auxiliary tag comprised of reference name, sorted read and mate fragmentation breakpoints, forward and reverse read barcodes, and read strand.

In some embodiments, the sequencing data is analysed using a variant calling algorithm that uses different subsets of read flags and tags (including read and mate coordinates, optical duplicate flag, UMI sequence(s), MID sequence(s), alignment scores, secondary alignment scores and others). In this case, analysis of sequencing data compares the probability of observing data under two models. The first is a null model specifying the distribution of sequencing artifacts. The second is a model allowing for true variants. In this case, a variant is called if the probability under the alternative model exceeds that of the null model. In some embodiments, a panel of pre-characterised samples can help to model the error distribution for the first model.

In some embodiments, auxiliary tags can be used to identify reads that likely derive from the same input molecule, and/or same strand of the same input molecule. In some embodiments, consensus base quality scores can be derived from reads that share the same auxiliary tag.

In some embodiments, variants are identified using an artificial intelligence algorithm such as convolutional neural networks.

In some embodiments, sequencing data can be further filtered to remove artefacts. Example filters include the number of mismatches present on a given sequencing read; the alignment score and next best alignment score; base quality scores or consensus base quality scores; the minimum number of reads covering a given variant site; the position of the variant within the sequencing read; whether reads are 5′ clipped; whether reads are improper pairs; whether reads contain indels; and the variant allele fraction of a given variant. In some embodiments, regions of the genome that include common SNPs, or are prone to alignment artefacts are filtered. There are many other filters known to the person skilled in the art.

In some embodiments a control sample is sequenced to filter out variants. For example, DNA from buccal epithelial, or other tissue sources, could be sequenced to remove germline variants. In another embodiment, buffy coat or leukocyte DNA can be sequenced to filter out somatic mutations that derive from clonal haematopoiesis.

The compositions, methods, and kits of the invention find use in a diverse range of applications and setting. In some embodiments, they find use in any methodology where a sequence is desired to be detected in a sample. In some embodiments, they find use in any methodology where there is a desire to detect a minority (e.g., rare) sequence in a complex sample. In addition to the exemplary uses described above, a number of additional illustrative uses are provided below.

In some embodiments, the compositions, methods, and kits find use in the analysis and treatment of infectious diseases. The technology is of particular value for detection of low frequency mutations that may be present in a sample. For example, the technology finds use for the detection of low frequency mutations associated with treatment resistant (e.g., antibiotic resistant, anti-viral resistant, etc.) infectious diseases (e.g., HIV, tuberculosis, etc.). The technology further finds use in selective pulldown of bacterial or viral DNA or RNA for sequencing.

As noted above, the technology is particularly well suited to the analysis and/or enrichment of analytes in complex samples. One area of growing research and clinical interest is microbiome analysis where the technology finds use to provide much higher specificity selection of desired bacterial DNA for sequencing or other analysis.

The technology also finds use in high throughput analysis of many different samples as well as multiplex analysis. These benefits find use in a wide variety of genotyping applications, including forensic analysis, paternity/maternity testing, disease analysis (e.g., cancer, infectious disease), drug susceptibility testing, agriculture and food testing (e.g., to assist with selective breeding, to identify trace contaminants, etc.).

The technology finds use in synthetic nucleic acid (e.g., DNA) error correction. Synthetic nucleic acid is used in research, diagnostic, and clinical indications. It is often important to avoid or minimizing use of nucleic acid molecules having unintended or undesired sequences. The technology finds use in identification and isolation of desired molecules from undesired molecules.

Nucleic acid editing is emerging as an important process in research, synthetic biology, and clinical applications. For example, CRISPR/CAS editing of nucleic acids, and related processes, are emerging as important processes. Many of these editing techniques result in a mixed populations of molecules that include intended edited products, unedited products, and unintended edited products. The technology provided herein facilitates identification, selection, and isolation of intended edited products.

The technology also finds use in environmental monitoring. In addition to agricultural uses, the technology is particularly well suited to the analysis of environmental samples that may contain trace amounts of an analyte of interest. Such samples include, but are not limited to, analysis of native and invasive species of organisms, early detection of invasive species, air and water contamination, and ancient DNA analysis. Sample types include, but are not limited to, soil, water, snow, feces, mucus, gametes, shed skin, carcasses, hair, and air.

The technology finds use in the isolation of a desired subset of nucleic acid from a particular sample from other subsets. For example, the technology finds use in the isolation and analysis of chloroplast and mitochondrial genomes.

The technology finds use in cell line screening for engineered and natural cells. Such cell lines include, but are not limited to, cell cultures (primary and immortalized), stems cells (embryonic, induced pluripotent, de-differentiated, etc.), differentiated cells intended for cell therapies, ex vivo modified cells for research or clinical applications (e.g., CAR T cells), and genetically engineered cells.

The technology finds use to remove damaged or other undesired nucleic acid away from undamaged or desired nucleic acid. For example, the technology may be used to remove damaged DNA from a sample prior to methylation analysis.

The technology finds use in pre-implantation screening of cells (e.g., embryos, eggs, sperm), liposomes, exosomes, nucleic acid vectors (e.g., gene therapy vector), and the like prior to their administration to a subject. In some embodiments, the technology can be applied to culture media to screen without disturbing live cells.

The technology finds use in drug toxicity screening. The technology is particularly well suited to the identification of DNA damage, generation of mutations, methylation changes, and the like that may be associated with the use of particular drugs.

The technology finds use in fragmentomic analysis of nucleic acids. For example, probes may be used that reside over or are aligned with a breakpoint that associates a particular sequence with relevant correlated information (e.g., tissue of origin, association with diseases such as cancer, etc.).

The technology may be used in any application where nucleic acid complexity reduction is desired. For example, the technology may be used for whole genome complexity reduction. In some such embodiments, a restriction enzyme digestion or other nucleic acid fragmenting process is used followed by the step of pulling out only cleaved molecules using probes that match known end sequences.

The technology finds use in assessing microsatellite instability (MSI). Target nucleic acid molecules that differ in the presence of, number or, or nature of repeated nucleotides (e.g., GT/CA repeats) are enriched and/or identified in a sample. MSI is associated with a number of diseases and conditions including, but not limited to, colon cancer, gastric cancer, endometrium cancer, ovarian cancer, hepatobiliary tract cancer, urinary tract caner, brain cancer, and skin cancers.

The technology also finds use in assessing tumor mutational burden (TMB). TMB has emerged as a predictive biomarker for immune checkpoint therapy, among other uses. Currently, next-generation, whole-exome sequencing is employed to assess TMB or a gene panel that provides sequences of a subset of genes is assessed. Use of the technology provided herein allows for TMB assessments that are more sensitive and significantly less costly and burdensome.

The technology also finds use in assessing and quantitating mutational processes including those associated with cancer. In one embodiment, probes are designed to sample somatic variants across a range of trinucleotide contexts. In one example, this approach can be used to identify tissue of origin. In another example, this approach can be to monitor environmental exposure to chemicals or radiation.

The technology finds use in haplotying. Genomic information reported as haplotypes rather than genotypes is increasingly important for personalized medicine, as well as a wide variety of research applications. Haplotypes, that are more specific than less complex variants such as single nucleotide variants, also have applications in prognostics and diagnostics, in the analysis of tumors, and in typing tissue for transplantation. Presently, sequencing is the most common form of molecular haplotyping. The error rate of sequencing technologies presents a barrier to obtaining accurate information. The technology provided herein allows for efficient and highly accurate haplotying.

In some embodiments, assay components are designed to avoid particular polymorphisms (e.g., SNPs). This may be desirable to avoid unwanted carryover of off-target molecules or lack of recovery of on-target molecules. In some embodiments, probes are designed to not hybridize to regions containing known polymorphisms (e.g., SNPs). In some embodiments, probes are designed and/or capture is configured to target a strand of the target sequence not containing a polymorphism to be avoided. In some embodiments, multiple probes are employed for each target, each probe targeting a different allele (e.g., SNP allele). In some embodiments, universal bases (e.g., inosine) are added to probes corresponding to known polymorphism sites (e.g., SNP sites), which are digested (or not) as though there was a sequence match at the polymorphism position whether the position contained the polymorphism sequence or a wild-type sequence.

### EXAMPLES

### Example 1

### Sequenced Probe: Mutation-Specific

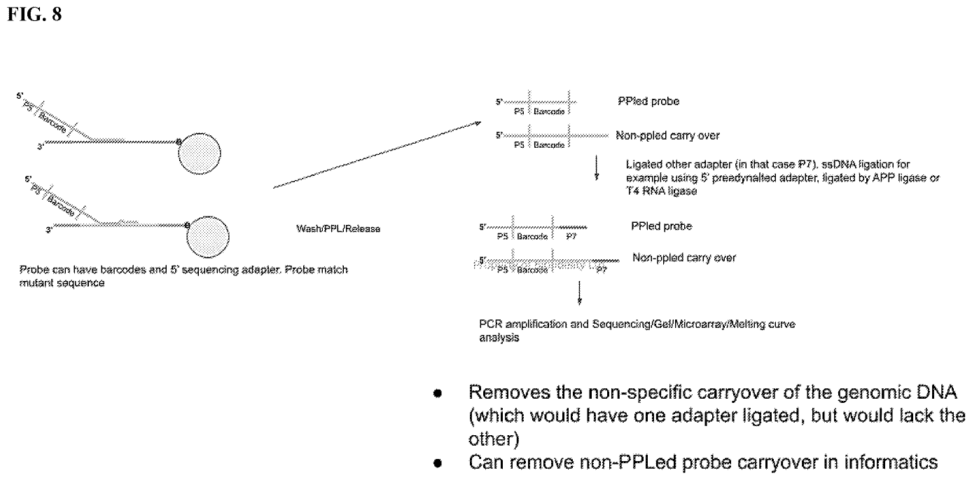

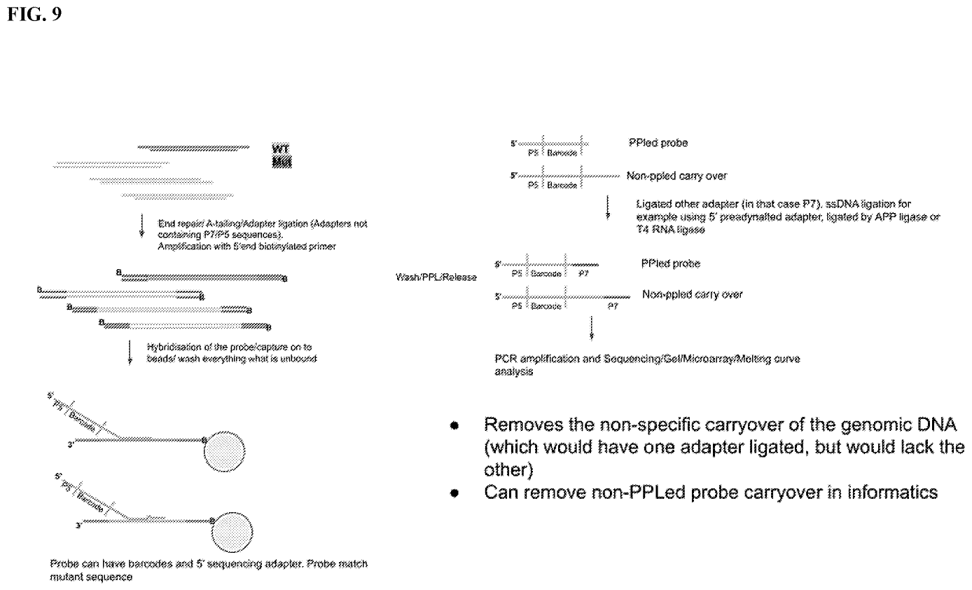

FIG. 8 provides a graphical overview of an experimental design used in this Example employing PPL as the digestion mechanism. FIG. 9 provides a graphical overview of a library preparation methodology that may be used employing PPL as the digestion mechanism. (WT=wild type; Mut=mutant).

### 1. Bead Preparation

a. Bead Blocking Step

Take 10 μL of beads per sample and put into a 1.5 mL eppendorf tube (ThermoFisher Dynabeads MyOne Streptavidin C1 cat. 65001). Up to 100 μL beads can be blocked with 1 mL of blocking solution.

Place the tube with beads on a magnet and wait until the beads separate and the solution is clear.

Remove storage buffer.

Add to the tube with beads a 1 mL blocking buffer (1×PBS, 0.1% Tween-20, 1 μg/mL t-RNA).

Rotate at room temperature (RT) for 30 min at 15 rpm.

b. Buffer Exchange

Spin down beads and place them on the magnet. Wait until the beads separate and the solution is clear.

Remove blocking solution.

Add 50 μL of the 2× Binding buffer (TrisHCl pH=7.5 40 mM, NaCl 1M, EDTA 2 mM, Tween20 0.02%) for every 10 μL of beads.

Mix the beads by vortexing for 5 seconds.

### 2. Oligo Hybridization

Prepare a dilution of oligonucleotides as follow:
ULTRAhyb™ Ultrasensitive Hybridization Buffer (cat no.: AM8670) 25 μL
Probe oligonucleotide 4 nM
WT or Mutant oligonucleotides mix 2 nM
Total volume: 50 μL
Incubate at 95° C. for 5 min and at 60° C. for 16 hours.

Probe oligonucleotide (SEQ ID NO: 1):
5′-AATGATACGGCGACCACCGAGATCTACACTATAGCCTGGGACC
CACTCCATCGAGATTTCTCTGTAGCTAG-3′

WT oligonucleotides mix comprises:

Forward strand (SEQ ID NO: 2):
5′-/5Biosg/CTGTTCAAACTGATGGGACCCACTCCATCGAGATT
TCACTGTAGCTAGACCAAAATCACCTATTTTTACTGTGAGGTCTTC
ATGAAGAAA-3′
Reverse strand (SEQ ID NO: 3):
5′-/5Biosg/TTTCTTCATGAAGACCTCACAGTAAAAATAGGTGA
TTTTGGTCTAGCTACAGTGAAATCTCGATGGAGTGGGTCCCATCAG
TTTGAACAG-3′

Mutant oligonucleotide mix comprises:

Forward strand (SEQ ID NO: 4):
5′-/5Biosg/CTGTTCAAACTGATGGGACCCACTCCATCGAGATT
TCTCTGTAGCTAGACCAAAATCACCTATTTTTACTGTGAGGTCTTC
ATGAAGAAA-3′
Reverse strand (SEQ ID NO: 5):
5′-/5Biosg/TTTCTTCATGAAGACCTCACAGTAAAAATAGGTGA
TTTTGGTCTAGCTACAGAGAAATCTCGATGGAGTGGGTCCCATCAG
TTTGAACAG-3′

Where/5Biosg/stands for biotin on the 5′ end.

### 3. Attachment Oligonucleotides to the Beads

Mix beads and oligonucleotides in the following ratio:

50 μL of beads from step 1.

50 μL of oligonucleotides from step 2.

Rotate for 30 min at RT at 15 rpm.

### 4. Bead Washing

I. After the attachment step is finished—spin down the samples and place them on a magnet and wait 7 minutes.

II. Remove all supernatants and add 100 μL of 1× Invitrogen™ NorthernMax™ Low Stringency Wash Buffer (cat. no.: AM8673) warmed to 65° C. Incubate sample for 3 min at 65° C.

III. Place samples on the magnet and wait 2 minutes.

IV. Repeat step II and III another two times.

V. Remove whole supernatant and take the samples out of the magnet and add 110 μL 1× Low TWEEN20 BFF6 buffer (Tris Acetate pH=7.0 10 mM, Potassium Acetate 30 mM, Magnesium Acetate 17.125 mM, TWEEN20 0.01%).

VI. Spin the samples down and place the samples on the magnet and wait 2 min.

VII. Transfer samples to the new 0.2 mL PCR strip.

VIII. Remove 1× Low TWEEN20 BFF6 buffer.

### 5. PPL Reaction

Add PPL mix to the beads kept at 4° C. PPL comprises the following:
1×BFF6-0.1% TWEEN20
20 U/mL Klenow (exo-)
2 U/mL Apyrase
+/−0.05 mM PPi
Total volume: 20 μL
Incubate at 40° C. for 10 min, 4° C. pause

### 6. TIPP Reaction

When the PPL reaction reaches 4° C., add 5 μL of TIPP mixture comprising:
1×BFF6-0.1% TWEEN20
16 U/mL TIPP
20 μL of mixture from step 5.
Total volume: 25 μL
Incubate at 37° C. for 5 min, 60° C. 10 min, 60° C. pause

### 7. Linker Ligation

When the reaction mixture from step 6 reaches 60° C. pause, move samples onto the magnet plate on the heated plate to 60° C. After beads will separate take 2.5 μL of supernatant and to the ligation mixture comprising:
1×NEBuffer 1 (10 mM Bis-Tris-Propane-HCl. 10 mM MgCl2, 1 mM DTT pH=7)
100 nM Linker
5 mM MnCl₂
20 pmol Thermostable 5′ App DNA/RNA Ligase
5% PEG8000
Total volume: 20 μL
Incubate at 65° C. for 60 min, 95° C. 2 min

Linker (SEQ ID NO: 6):
/5rApp/ATTACTCGATCTCGTATGCCGTCTTCTGCTTG/3SpC3/

### 8. Amplification

Take 4 μL of mixture from step 7 and add to following components:
1× Q5U buffer
dNTPs 0.4 mM
Primer mix 1 0.2 μM
Q5U polymerase 20 U/mL
UDG 10 U/mL
Total volume: 12.5 μL
Q5U buffer

Primer mix 1 comprises:

Forward primer (SEQ ID NO: 7):
5′-AATGATACGGCGACCACCGAGATCTACAC-3′
Reverse primer (SEQ ID: 8):
5′-CAAGCAGAAGACGGCATACGAGAT-3′

Place sample in the thermocycler and incubate with lid on 105° C.

| | | | |
|---|---|---|---|
| 1. UDG | 37° C. | 1 | min |
| 2. Int. denaturation | 98° C. | 1 | min |
| 3. Denaturation | 98° C. | 10 | sec |
| 4. Annealing | 63° C. | 15 | sec |
| 5. Elongation | 72° C. | 15 | sec |
| 6. Final elongation | 72° C. | 5 | min |

| | | |
|---|---|---|
| 7. Cool down | 4° C. | hold |

Step 3-5 repeated 30x

### 9. Gel Electrophoresis

Take 8 μL sample from step 8 and add 4 μL of ThermoFisher DNA Gel Loading Dye (6×) (cat. no.: R0611).

Run samples in 12% polyacrylamide gel at 90V for 150 min using Bio-rad Mini-PROTEAN Tetra Cell tanks.

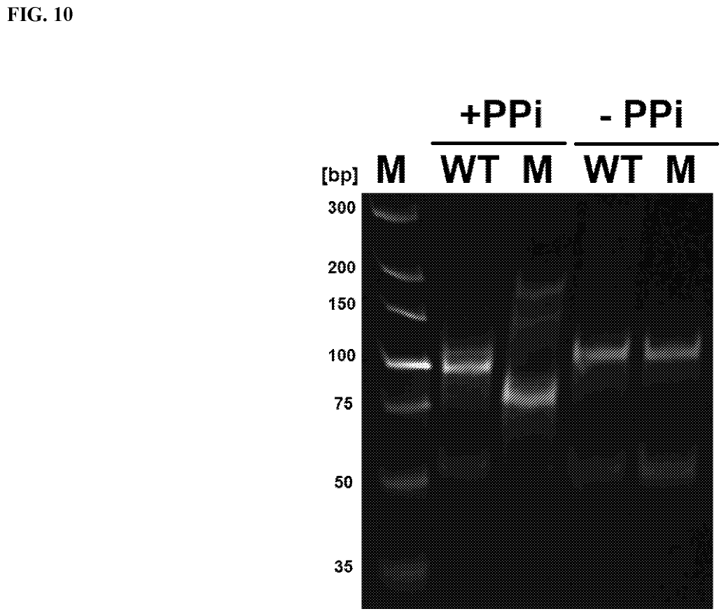

Visualize gel using Axygen® Gel Documentation System GD-1000. The image of the gel is shown in FIG. 10, showing successful detection of BRAF V600E mutation. The gel shows PPi dependent shortening of the probe in the presence of V600E mutation in the target molecule. M—Invitrogen™ Ultra Low Range DNA Ladder (Cat. no: 10597012), WT—wild type molecule, M—mutant molecule containing V600E mutation, PPi—pyrophosphate ion.

### Example 2

### Sequenced Probe: Mutation-Specific at Varying Concentrations

### 1. Bead Preparation

a. Bead Blocking Step

Take 10 μL of beads per sample and put into a 1.5 mL eppendorf tube (ThermoFisher Dynabeads MyOne Streptavidin C1 cat. 65001). Up to 100 μL beads can be blocked with 1 mL of blocking solution.

Place the tube with beads on a magnet and wait until the beads separate and the solution is clear.

Remove storage buffer.

Add to the tube with beads a 1 mL blocking buffer (1×PBS, 0.1% TWEEN20, 1 μg/mL t-RNA).

Rotate at RT for 30 min at 15 rpm.

b. Buffer Exchange

Spin down beads and place them on a magnet. Wait until the beads separate and the solution is clear.

Remove blocking solutions.

Add 50 μL of the 2× Binding buffer (TrisHCl pH=7.5 40 mM, NaCl 1M, EDTA 2 mM, TWEEN20 0.02%) for every 10 μL of beads.

Mix the beads by vortexing for 5 seconds.

### 2. Oligo Hybridization

Prepare dilution of oligonucleotides as follow
ULTRAhyb™ Ultrasensitive Hybridization Buffer (cat no.: AM8670) 25 μL
Probe oligonucleotide 4 nM
WT oligonucleotides mix 0/2 nM
Mutant oligonucleotides mix 0.2-2 nM
Total volume: 50 μL
Incubate at 95° C. for 5 min and at 60° C. for 16 hours.

Probe oligonucleotide (SEQ ID NO: 9):
5′-GTCTCGTGGGCTCGGAGATGTGTATAAGAGACAGGGGACCCAC
TCCATCGAGATTTCTCTGTAGCTAG-3′

WT oligonucleotides mix comprises:

Forward strand (SEQ ID NO: 10):
5′-/5Biosg/CTGTTCAAACTGATGGGACCCACTCCATCGAGATTT
CACTGTAGCTAGACCAAAATCACCTATTTTTACTGTGAGGTCTTCAT
GAAGAAA-3′
Reverse strand (SEQ ID NO: 11):
5′-/5Biosg/TTTCTTCATGAAGACCTCACAGTAAAAATAGGTGAT
TTTGGTCTAGCTACAGTGAAATCTCGATGGAGTGGGTCCCATCAGTT
TGAACAG-3′

Mutant oligonucleotide mix comprises:

Forward strand (SEQ ID NO: 12):
5′-/5Biosg/CTGTTCAAACTGATGGGACCCACTCCATCGAGATTT
CTCTGTAGCTAGACCAAAATCACCTATTTTTACTGTGAGGTCTTCAT
GAAGAAA-3′
Reverse strand (SEQ ID NO: 13):
5′-/5Biosg/TTTCTTCATGAAGACCTCACAGTAAAAATAGGTGAT
TTTGGTCTAGCTACAGAGAAATCTCGATGGAGTGGGTCCCATCAGTT
TGAACAG-3′

Where/5Biosg/stands for biotin on the 5′ end.

### 3. Attachment Oligonucleotides to the Beads

Mix beads and oligonucleotides in the following ratio:

50 μL of beads from step 1.

50 μL of oligonucleotides from step 2.

Rotate for 30 min at RT at 15 rpm.

### 4. Bead Washing

I. After the attachment step is finished—spin down the samples and place them on the magnet and wait 7 minutes.

II. Remove all supernatants and add 100 μL of 1× Invitrogen™ NorthernMax™ Low Stringency Wash Buffer (cat. no.: AM8673) warmed to 65° C. Incubate sample for 3 min at 65° C.

III. Place samples on the magnet and wait 2 minutes.

IV. Repeat step II and III another two times.

V. Remove whole supernatant and take the samples out of the magnet and add 110 μL 1× Low TWEEN20 BFF6 buffer (Tris Acetate pH=7.0 10 mM, Potassium Acetate 30 mM, Magnesium Acetate 17.125 mM, TWEEN20 0.01%).

VI. Spin the samples down and place the samples on the magnet and wait 2 min.

VII. Transfer samples to the new 0.2 mL PCR strip.

VIII. Remove 1× Low Tween20 BFF6 buffer.

### 5. PPL Reaction

Add PPL mix to the beads kept at 4° C. PPL mixture comprises the following:
1×BFF6-0.1% TWEEN20
20 U/mL Klenow (exo-)
2 U/mL Apyrase
+/−0.05 mM PPi
Total volume: 20 μL
Incubate at 40° C. for 10 min, 4° C. pause

### 6. TIPP Reaction

When the PPL reaction reaches 4° C., add 5 μL of TIPP mixture comprising:
1×BFF6-0.1% TWEEN20
16 U/mL TIPP
20 μL of mixture from step 5.
Total volume: 25 μL
Incubate at 37° C. for 5 min, 60° C. 10 min, 60° C. pause

### 7. Linker Adenylation

Linker adenylation mixture comprises:
1× Adenylation Reaction buffer
40 μM Linker
1 mM ATP
40 μM Mth RNA ligase
Total volume: 20 μL
Incubate at 65° C. for 60 min, 95° C. 2 min

Linker (SEQ ID NO: 14):
/5Phos/ATTACTCGATCTCGTATGCCGTCTTCTGCTTG/3SpC3/

### 8. Linker Ligation

When the reaction mixture from step 6 reaches 60° C. pause, move samples onto the magnet plate on the heated plate to 60° C. After beads will separate take 2.5 μL of supernatant and to the ligation mixture comprises:
1×NEBuffer 1 (10 mM Bis-Tris-Propane-HCl. 10 mM MgCl₂, 1 mM DTT pH=7)
0.75 nM Linker from step 7.
5 mM MnCl₂
20 pmol Thermostable 5′ App DNA/RNA Ligase
5% PEG8000
Total volume: 20 μL
Incubate at 65° C. for 60 min, 95° C. 2 min

### 9. Amplification

Take 4 μL of mixture from step 7 and add to following components:
1×QSU buffer
dNTPs 0.4 mM
Primer mix 2 0.2 μM
QSU polymerase 20 U/mL
UDG 10 U/mL
Total volume: 12.5 μL
QSU buffer

Primer mix 2 comprises:

Forward primer (SEQ ID NO: 15):
5′-AATGATACGGCGACCACCGAGATCTACAC-3′
Reverse primer (SEQ ID NO: 16):
5′-CAAGCAGAAGACGGCATACGAGAT-3′

Place sample in the thermocycler and incubate with lid on 105° C.

| | | | |
|---|---|---|---|
| 1. UDG | 37° C. | 1 | min |
| 2. Int. denaturation | 98° C. | 1 | min |
| 3. Denaturation | 98° C. | 10 | sec |
| 4. Annealing | 63° C. | 15 | sec |
| 5. Elongation | 72° C. | 15 | sec |
| 6. Final elongation | 72° C. | 5 | min |

| | | |
|---|---|---|
| 7. Cool down | 4° C. | hold |

Step 3-5 repeated 30x

### 10. Gel Electrophoresis

Take 8 μL sample from step 8 and add 4 μL of ThermoFisher DNA Gel Loading Dye (6×) (cat. no.: R0611).

Run samples in 12% polyacrylamide gel at 90V for 150 min using Bio-rad Mini-PROTEAN Tetra Cell tanks.

Visualize gel using Axygen® Gel Documentation System GD-1000. Gel results showed detection of BRAF V600E mutation. The gel showed PPi dependent shortening of the probe in the presence of V600E mutation in the target molecule at 50%, 10%, and 1% concentration relative to wild-type sequence.

### Example 3

### Detection of EGFR Exon 20 T790M

### 1. Bead Preparation

a. Bead Blocking Step

Take 1 μL of beads per sample and put into a 1.5 mL eppendorf tube (ThermoFisher Dynabeads MyOne Streptavidin C1 cat. 65001). Up to 100 μL beads can be blocked with 1 mL of blocking solution.

Place the tube with beads on a magnet and wait until the beads separate and the solution is be clear.

Remove storage buffer.

Add to the tube with beads a 1 mL blocking buffer (1×PBS, 0.1% TWEEN20, 1 μg/mL t-RNA).

Rotate at RT for 30 min at 15 rpm.

b. Buffer Exchange

Spin down beads and place them on the magnet. Wait until the beads separate and the solution is clear.

Remove blocking solutions.

Add 50 μL of the 2× Binding buffer (TrisHCl pH=7.5 40 mM, NaCl 1M, EDTA 2 mM, TWEEN20 0.02%) for every 1 μL of beads.

Mix the beads by vortexing for 5 seconds.

### 2. Oligo Hybridization

Prepare dilution of oligonucleotides as follow:
ULTRAhyb™ Ultrasensitive Hybridization Buffer (cat no.: AM8670) 25 μL
Probe oligonucleotide 2 pM
WT oligonucleotides mix 180-200 fM
Mutant oligonucleotides mix 0.2-20 fM
Total volume: 50 μL
Incubate at 95° C. for 5 min and at 60° C. for 16 hours.

Probe oligonucleotide (SEQ ID NO: 17):
5′-CACATCTAGAGCCACCAGCGGCATAGTAATATAGCCTCCAGGAGG
CAGCCGAAGGGCATGAGCTGCGTGATG-3′

WT oligonucleotides mix comprises:

Forward strand (SEQ ID NO: 18):
5′-ACGCGTGGTTACAGTCTTGCGCATCTGCCTCACCTCCACCGTGCA
GCTCATCACGCAGCTCATGCCCTTCGGCTGCCTCCTGGACTATGT/
3Biosg/-3′
Reverse strand (SEQ ID NO: 19):
5′-ACATAGTCCAGGAGGCAGCCGAAGGGCATGAGCTGCGTGATGAGC
TGCACGGTGGAGGTGAGGCAGATGCGCAAGACTGTAACCACGCGT/
3Biosg/-3′

Mutant oligonucleotide mix comprises:

Forward strand (SEQ ID NO: 20):
5′-GTGCTCCCCCGCCAATTCCATCTGCCTCACCTCCACCGTGCAGCT
CATCATGCAGCTCATGCCCTTCGGCTGCCTCCTGGACTATGTCC/
3Biosg/-3′
Reverse strand (SEQ ID NO: 21):
5′-GGACATAGTCCAGGAGGCAGCCGAAGGGCATGAGCTGCATGATGA
GCTGCACGGTGGAGGTGAGGCAGATGGAATTGGCGGGGGAGCAC/
3Biosg/-3′

Where/5Biosg/stands for biotin on the 5′ end.

### 3. Attachment Oligonucleotides to the Beads

Mix beads and oligonucleotides in the following ratio:

50 μL of beads from step 1.

50 μL of oligonucleotides from step 2.

Rotate for 30 min at RT at 15 rpm.

### 4. 1st Bead Washing

I. After the attachment step is finished—spin down the samples and place them on the magnet and wait 7 minutes.

II. Remove whole supernatant and take the samples out of the magnet and add 100 μL 1× Low TWEEN20 BFF6 buffer (Tris Acetate pH=7.0 10 mM, Potassium Acetate 30 mM, Magnesium Acetate 17.125 mM, TWEEN20 0.01%).

III. Spin the samples down and place the samples on the magnet and wait 2 min.

IV. Repeat steps II and III one more time.

V. Transfer samples to the new 0.2 mL PCR strip or 96 well PCR plate.

VI. Remove 1× Low TWEEN20 BFF6 buffer.

### 5. PPL Reaction

Add PPL mix to the beads kept at 4° C. PPL mixture comprises the following:
1×BFF6-0.1% TWEEN20
20 U/mL Klenow (exo-)
2 U/mL Apyrase
+/−0.05 mM PPi
Total volume: 20 μL
Incubate at 40° C. for 10 min, 4° C. pause

### 6. TIPP Reaction

When the PPL reaction reaches 4° C., add 5 μL of TIPP mixture comprising:
1×BFF6-0.1% TWEEN20
16 U/mL TIPP
20 μL of mixture from step 5.
Total volume: 25 μL
Incubate at 37° C. for 5 min, 60° C. 10 min, 60° C. pause

### 7. 2nd Bead Washing

I. After the attachment step is finished—spin down the samples and place them on the magnet and wait 7 minutes.

II. Remove all supernatants and add 100 μL of 1× Invitrogen™ NorthernMax™ Low Stringency Wash Buffer (cat. no.: AM8673) warmed to 65° C. Incubate sample for 3 min at 65° C.

III. Place samples on the magnet and wait 2 minutes.

IV. Repeat step II and III another two times.

V. Remove the whole supernatant and take the samples out of the magnet and add 100 μL 1×Q5U buffer.

VI. Spin the samples down and place the samples on the magnet and wait 2 min.

VII. Transfer samples to the new 0.2 mL PCR strip or 96-well PCR plate.

VIII. Remove 1×Q5U buffer.

### 8. Bead Amplification

Add to the beads amplification mixture consists of:
1×QSU buffer
dNTPs 0.4 mM
WT or mutant specific primer mix 5 μM
QSU polymerase 20 U/mL
UDG 10 U/mL
Total volume: 12.5 μL

WT specific primer mix comprises:

Forward primer (SEQ ID NO: 22):
5′-CACATCTAGAGCCACCAGCGGCATAGTAA-3′
Reverse primer (SEQ ID NO: 23):
5′-ACGCGTGGTTACAGTCTTGCG-3′

Mutant specific primer mix comprises:

Forward primer (SEQ ID NO: 24):
5′-CACATCTAGAGCCACCAGCGGCATAGTAA-3′
Reverse primer (SEQ ID NO: 25):
5′-GTGCTCCCCCGCCAATTC-3′

Place sample in the thermocycler and incubate with lid on 105° C.

| | | | |
|---|---|---|---|
| 1. UDG | 37° C. | 1 | min |
| 2. Extension | 63° C. | 10 | min |
| 3. Int. denaturation | 98° C. | 1 | min |
| 4. Denaturation | 98° C. | 10 | sec |
| 5. Annealing | 63° C. | 15 | sec |
| 6. Elongation | 72° C. | 15 | sec |
| 7. Final elongation | 72° C. | 5 | min |

| | | |
|---|---|---|
| 8. Cool down | 4° C. | hold |

Step 3-5 repeated 12x

9. dPCR Amplification

Add to the beads amplification mixture comprising:
1×Q5U buffer
dNTPs 0.4 mM
EvaGreen dye 2×
AlexaFluor dye 0.0003 μg/μL
TWEEN20 0.2%
WT or mutant specific primer mix 5 μM
Q5U polymerase 20 U/mL
UDG 10 U/mL
Template from step 8 2 μL
Total volume: 12 μL
Place sample in the QIAcuity Digital PCR System with lid on 105° C.

| | | | |
|---|---|---|---|
| 1. Int. denaturation | 98° C. | 1 | min |
| 2. Denaturation | 98° C. | 10 | sec |
| 3. Annealing | 63° C. | 15 | sec |
| 4. Elongation | 72° C. | 15 | sec |
| 5. Final elongation | 72° C. | 5 | min |

| | | |
|---|---|---|
| 6. Cool down | 35° C. | hold |

Step 2-4 repeated 30x

Take a picture of the partitions in green and yellow channels with exposure duration 600 and 700 ms respectively and gain 6 and 8 respectively.

We claim:

1. A method comprising: contacting a sample with a probe that differs in complementarity to a target region of a first nucleic acid molecule relative to a second nucleic acid molecule in the sample; optionally activating probe hybridized to said first nucleic acid molecule by selectively modifying said probe hybridized to said first nucleic acid molecule relative to probe hybridized to said second nucleic acid molecule; selectively digesting probe hybridized to said first or said second nucleic acid molecule relative to the other; and sequencing at least a portion of probe that was hybridized to said first nucleic acid molecule.

2. The method of claim 1, wherein said first and second nucleic acid molecules comprise end-repaired nucleic acid molecules.

3. The method of claim 1 or 2, wherein said first and second nucleic acid molecules are A-tailed nucleic acid molecules.

4. The method of any of claims 1 to 3, wherein said first and second nucleic acid molecules comprise an adapter sequence.

5. The method of any of claims 1 to 4, wherein said first and second nucleic acid molecules are amplified.

6. The method of any of claims 1 to 5, wherein said probe is activated by contact with a cleavage agent.

7. The method of claim 6, wherein said cleavage agent is selected from the group consisting of a restriction endonuclease, a flap endonuclease, a mismatch repair enzyme, an RNase, a Cas protein, an argonaute family enzyme, a DNA-formamidopyrimidine glycosylase (Fpg), an apurinic/apyrimidinic (AP) endonuclease (APE 1), and a chemical cleavage agent.

8. The method of any of claims 1 to 7, wherein said digesting comprises contacting said probe with an exonuclease or endonuclease.

9. The method of claim 8, wherein said exonuclease is a 3′ to 5′ exonuclease.

10. The method of claim 8, wherein said exonuclease is a 5′ to 3′ exonuclease.

11. The method of any of claims 1 to 7, wherein said digesting comprises a pyrophosphorolysis reaction.

12. The method of any of claims 1 to 11, wherein said probe or said first and/or second nucleic acid molecules comprises a binding moiety at its 3′ end, 5′ end, or internally, said binding moiety optionally a biotin.

13. The method of any of claims 1 to 11, wherein said probe or said first and/or second nucleic acid molecules comprises a linker that can be hybridized to a complementary sequence bound to a solid support.

14. The method of any of claims 1 to 13, wherein said first or second nucleic acid molecule comprises or is derived from a molecule comprising one or more methylated nucleotides.

15. The method of any of claims 1 to 14, wherein said probe or said first and/or second nucleic acid molecules comprises a blocking group.

16. The method of any of claims 1 to 15, wherein said probe comprises an adaptor sequence.

17. The method of claim 6, wherein said activated probe undergoes extension.

18. The method of claim 17, wherein said extension is performed using nucleotides including a binding moiety.

19. The method of any of claims 1-18 wherein said first or second nucleic acid molecule is attached to a solid support prior to said digesting.

20. The method of any of claims 1-18, wherein said first or second nucleic acid molecule is attached to a solid support after said digesting.

21. The method of any of claims 1 to 20, further comprising the step of capturing said probe or said first and/or second nucleic acid molecules on a surface prior to said digesting.

22. The method of claim 21, wherein said surface comprises a bead.

23. The method of claim 21, further comprising the step of differentially releasing said probe hybridized to said first nucleic acid molecule or said second nucleic acid molecule.

24. The method of claim 23, wherein said releasing comprises increasing temperature.

25. The method of claim 23, wherein said releasing comprises changing pH.

26. The method of claim 23, wherein said releasing comprises changing salt concentration.

27. The method of claim 23, wherein said releasing comprises said digesting.

28. The method of any of claims 1 to 27, further comprising the step of detecting said first or second nucleic acid molecule.

29. The method of claim 28, wherein said detecting comprises sequencing a probe molecule that was hybridized to said first or second nucleic acid molecule.

30. The method of claim 29, further comprising sequencing said first or second nucleic acid molecule.

31. The method of any of claims 1 to 30, wherein said probe comprises a base that is complementary to a position in said first nucleic acid molecule and is mismatched with a corresponding position in said second nucleic acid molecule.

32. The method of claim 31, wherein said digesting comprises contacting probe hybridized to said first and second nucleic acid molecules with an enzyme that preferentially digests complementary strands over non-complementary strands or with an enzyme that preferentially digests non-complementary strands over complementary strands.

33. The method of any of claims 1 to 32, wherein said probe comprises a 3′ end, 5′ end, or internal blocking group.

34. The method of claim 33, wherein said probe is activated using a cleavage agent that removes said blocking group from probe hybridized to said first nucleic acid molecule but not from probe hybridized to said second nucleic acid molecule.

35. The method of claim 34, wherein said digesting comprises contacting probe with a nuclease that cleaves probe lacking a blocking group but does not cleave probe having a blocking group.

36. A kit comprising reagents sufficient for conducting the method of any of claims 1 to 35 on a sample comprising said first and second nucleic acid molecules.

37. A method comprising: contacting a sample with a probe that differentially hybridizes to a target region of a first nucleic acid molecule relative to a second nucleic acid molecule in the sample, wherein said first or second nucleic acid molecule comprises a gene fusion or indel; optionally activating probe hybridized to said first nucleic acid molecule by selectively modifying said probe hybridized to said first nucleic acid molecule relative to probe hybridized to said second nucleic acid molecule; selectively digesting probe hybridized to said first or said second nucleic acid molecule relative to the other; and detecting said first and/or second nucleic acid molecule.

38. The method of claim 37, wherein said probe differentially hybridizes to said first nucleic acid molecule relative to said second nucleic molecule based on differential complementarity of said probe to said first nucleic acid relative to said second nucleic acid.

39. The method of claim 37, wherein said probe differentially hybridizes to said first nucleic acid molecule relative to said second nucleic molecule based on the presence of a blocking oligonucleotide hybridized to said first nucleic acid molecule but not said second nucleic acid molecule, wherein a 3′ region of said probe is blocked by said blocking oligonucleotide.

40. The method of claim 39, wherein said blocking oligonucleotide selectively hybridizes to wild-type sequence relative to sequence comprising a gene fusion or indel.

41. The method of any of claims 37 to 40, wherein said first or second nucleic acid molecule is enriched relative to the other following said digesting.

42. The method of any of claims 37 to 41, wherein said digestion comprises pyrophosphorolysis.

43. The method of claim 42, wherein said digesting comprises contacting said sample with said probe, a pyrophosphorolysing enzyme, and a source of pyrophosphate ion.

44. The method of any of claims 37 to 43, wherein said probe, said first nucleic acid molecule, and/or said second nucleic acid molecule is attached to solid support.

45. The method of claim 44, wherein said probe, said first nucleic acid molecule, and/or said second nucleic acid molecule is attached to the solid support prior to said digesting.

46. The method of any of claims 37 to 45, wherein said probe comprises a 5′ tail region that is not complementary to said first or second nucleic acid molecules.

47. The method of claim 46, wherein said 5′ tail region is complementary to a capture oligonucleotide attached to a solid support.

48. The method of claim 44, wherein said probe, said first nucleic acid molecule, and/or said second nucleic acid molecule comprises a capture moiety.

49. The method of claim 48, wherein said capture moiety is biotin and said solid support comprises streptavidin.

50. The method of claim 48, wherein said solid support is a bead.

51. The method of claim 50, wherein the bead is a magnetic or paramagnetic bead.

52. The method of any of claims 37 to 51, wherein one or more wash steps are performed between one or more of the steps.

53. The method of any of claims 37 to 52, wherein said sample is an adaptor tagged library of nucleic acid molecules.

54. The method of any of claims 37 to 53, wherein said detecting comprises a nucleic acid amplification step.

55. The method of any of claims 37 to 54, wherein said detecting comprises sequencing.

56. The method of claim 55, wherein said sequencing comprises Next Generation Sequencing (NGS).

57. The method of any of claims 37 to 56, wherein multiple different probe oligonucleotides are employed, each with a different sequence designed to anneal to a different target sequence.

58. The method of any of claims 37 to 57, wherein said sample is, or is derived from, a human blood or tissue sample.

59. The method of any of claims 37 to 58, wherein said probe comprises a ‘5 region complementary to a portion of a wild-type sequence and a 3’ portion not complementary to said wild-type sequence.

60. A kit comprising reagents sufficient, necessary, or useful for conducting the method of any of claims 37 to 59 on a sample comprising said first and second nucleic acid molecules.

61. The kit of claim 60, comprising one or more or all of: said probe, an activation reagent, a digestion reagent, a solid support, a buffer, a capture reagent, an amplification reagents, a sequencing reagent, a positive control, a negative control, a sequencing adapter, a ligase, a library preparation reagent, a crowding agent, and a detergent.

62. Use of a kit of claim 60 or 61.


SEQUENCE LISTING (25 sequences)

SEQ ID NO: 1 - DNA, 71 bp, synthetic construct
  source 1..71: mol_type = other DNA
aatgatacggcgaccaccgagatctacactatagcctgggacccactccatcgagatttctctgtagctag

SEQ ID NO: 2 - DNA, 90 bp, synthetic construct
  source 1..90: mol_type = other DNA
ctgttcaaactgatgggacccactccatcgagatttcactgtagctagaccaaaatcacctatttttactgtgaggtcttcatgaagaaa

SEQ ID NO: 3 - DNA, 90 bp, synthetic construct
  source 1..90: mol_type = other DNA
tttcttcatgaagacctcacagtaaaaataggtgattttggtctagctacagtgaaatctcgatggagtgggtcccatcagtttgaacag

SEQ ID NO: 4 - DNA, 90 bp, synthetic construct
  source 1..90: mol_type = other DNA
ctgttcaaactgatgggacccactccatcgagatttctctgtagctagaccaaaatcacctatttttactgtgaggtcttcatgaagaaa

SEQ ID NO: 5 - DNA, 90 bp, synthetic construct
  source 1..90: mol_type = other DNA
tttcttcatgaagacctcacagtaaaaataggtgattttggtctagctacagagaaatctcgatggagtgggtcccatcagtttgaacag

SEQ ID NO: 6 - DNA, 32 bp, synthetic construct
  source 1..32: mol_type = other DNA
attactcgatctcgtatgccgtcttctgcttg

SEQ ID NO: 7 - DNA, 29 bp, synthetic construct
  source 1..29: mol_type = other DNA
aatgatacggcgaccaccgagatctacac

SEQ ID NO: 8 - DNA, 24 bp, synthetic construct
  source 1..24: mol_type = other DNA
caagcagaagacggcatacgagat

SEQ ID NO: 9 - DNA, 68 bp, synthetic construct
  source 1..68: mol_type = other DNA
gtctcgtgggctcggagatgtgtataagagacaggggacccactccatcgagatttctctgtagctag

SEQ ID NO: 10 - DNA, 90 bp, synthetic construct; identical to SEQ ID NO: 2
  source 1..90: mol_type = other DNA

SEQ ID NO: 11 - DNA, 90 bp, synthetic construct; identical to SEQ ID NO: 3
  source 1..90: mol_type = other DNA

SEQ ID NO: 12 - DNA, 90 bp, synthetic construct; identical to SEQ ID NO: 4
  source 1..90: mol_type = other DNA

SEQ ID NO: 13 - DNA, 90 bp, synthetic construct; identical to SEQ ID NO: 5
  source 1..90: mol_type = other DNA

SEQ ID NO: 14 - DNA, 32 bp, synthetic construct; identical to SEQ ID NO: 6
  source 1..32: mol_type = other DNA

SEQ ID NO: 15 - DNA, 29 bp, synthetic construct
  source 1..29: mol_type = other DNA
aatgatacggcgaccaccgagatctacac

SEQ ID NO: 16 - DNA, 24 bp, synthetic construct
  source 1..24: mol_type = other DNA
caagcagaagacggcatacgagat

SEQ ID NO: 17 - DNA, 72 bp, synthetic construct
  source 1..72: mol_type = other DNA
cacatctagagccaccagcggcatagtaatatagcctccaggaggcagccgaagggcatgagctgcgtgatg

SEQ ID NO: 18 - DNA, 90 bp, synthetic construct
  source 1..90: mol_type = other DNA
acgcgtggttacagtcttgcgcatctgcctcacctccaccgtgcagctcatcacgcagctcatgcccttcggctgcctcctggactatgt

SEQ ID NO: 19 - DNA, 90 bp, synthetic construct
  source 1..90: mol_type = other DNA
acatagtccaggaggcagccgaagggcatgagctgcgtgatgagctgcacggtggaggtgaggcagatgcgcaagactgtaaccacgcgt

SEQ ID NO: 20 - DNA, 89 bp, synthetic construct
  source 1..89: mol_type = other DNA
gtgctcccccgccaattccatctgcctcacctccaccgtgcagctcatcatgcagctcatgcccttcggctgcctcctggactatgtcc

SEQ ID NO: 21 - DNA, 89 bp, synthetic construct
  source 1..89: mol_type = other DNA
ggacatagtccaggaggcagccgaagggcatgagctgcatgatgagctgcacggtggaggtgaggcagatggaattggcgggggagcac

SEQ ID NO: 22 - DNA, 29 bp, synthetic construct
  source 1..29: mol_type = other DNA
cacatctagagccaccagcggcatagtaa

SEQ ID NO: 23 - DNA, 21 bp, synthetic construct
  source 1..21: mol_type = other DNA
acgcgtggttacagtcttgcg

SEQ ID NO: 24 - DNA, 29 bp, synthetic construct
  source 1..29: mol_type = other DNA
cacatctagagccaccagcggcatagtaa

SEQ ID NO: 25 - DNA, 18 bp, synthetic construct
  source 1..18: mol_type = other DNA
gtgctcccccgccaattc